(19) United States
(12) Patent Application Publication
(10) Pub. No.: US 2010/0016983 A1
(43) Pub. Date: Jan. 21, 2010

(54) HIP PROTECTOR IMPLANT

(76) Inventor: Arno Smit, White Rock (CA)

Correspondence Address: SCHUBERT OSTERRIEDER & NICKELSON PLLC, 6013 CANNON MTN DR, S14, AUSTIN, TX, 78749, US

(21) Appl. No.: 12/296,994

(22) PCT Filed: Apr. 13, 2007
(86) PCT No.: PCT/CA07/00626

§ 371 Date: Aug. 25, 2009

Related U.S. Application Data
(60) Provisional application No. 60/791,471, filed on Apr. 13, 2006.

(51) Int. Cl. A61F 2/32

(52) U.S. Cl. 623/22.4

(57) ABSTRACT

An implant system is described to decrease the risk of hip fracture in humans determined to be at increased risk for hip fracture, over a time span well over several years after implantation. This implant increases the size of the contact area on the proximal femur at the time of a fall, decreasing contact pressures and contact stresses. The implant may be able to absorb energy on impact or cause an increase of energy absorption by the soft tissues, thus decreasing the energy transfer to the proximal femur at the time of a fall. The implant may also strengthen the proximal femur, while minimizing stress shielding of the surrounding bone. In addition, it minimizes the risk of displacement should fracture occur, thus minimizing the risk of formal fracture surgery. The method of application minimizes risks associated with initial application of the implant.

This application claims the benefit of U.S. Patent Application No. 60/791,471, which is hereby incorporated by reference.

### FIELD OF THE INVENTION

The invention relates to implants for preventing bone fractures, and more particularly for preventing hip fractures.

### BACKGROUND OF THE INVENTION

Low to moderate energy fractures of the hip, such as those caused by a fall from a standing or sitting position, pose a significant clinical and social problem. The majority of these fractures occur among the elderly, although there are additional risk groups, such as patients with underlying primary bone disorders, e.g. osteomalacia or osteogenesis imperfecta; patients with neurological disorders, either through increased fall risk, e.g. Parkinson's disease, or through secondary bone disorders, e.g. disuse osteopenia associated with paraplegia; or patients with osteomalacia as a result of the use of certain pharmacological agents, e.g. anticonvulsants. It is estimated that among the elderly, approximately 90% of hip fractures are due to a fall, and the remaining 10% of hip fractures are insufficiency fractures caused by a mechanical overload in activities such as normal walking. When localized bone destruction occurs as part of a generalized disorder such as cancer, a high risk of fracture, limited to the area(s) of bone destruction, may develop.

Preventive strategies have been focused on the elderly. Many risk factors for hip fractures have been identified, including increased fall risk due to lack of balance; sub-optimal musculoskeletal conditioning; low body mass index with resultant paucity of soft tissue coverage of the greater trochanter and increased energy transmission to the bone with a fall; and impaired bone quality, through loss of mineral content as well as disruption of bony architecture.

Strategies employed for prevention of hip fractures in the elderly usually involve lifestyle modification, through musculoskeletal conditioning, balance exercises, diet optimization and avoidance of risk factors for osteoporosis. In addition, multiple pharmacological interventions exist, with varying degrees of documented success: bone density loss can be attenuated or reversed to some degree, resulting in diminished fracture risk. However, once significant bony architecture has been lost, it is unlikely that significant benefit can be achieved from pharmacological treatment alone. In addition, ongoing pharmacological treatment exposes patients to ongoing side effects of the medication.

The role of surgery as a preventive strategy has been limited to individuals with very high fracture risk, such as the presence of localized destructive bone lesions, or in extreme cases of generalized impaired bone quality, such as osteogenesis imperfecta.

Surgical re-enforcement of weakened bone with rigid implants will initially increase the strength of the bone/implant combination. This may be sufficient if the time span during which fractures can occur is relatively limited, such as in the case of a localized destructive lesion caused by cancer, leading to a decreased life expectancy. However, given time, the stress shielding of the surrounding bone in the presence of a rigid implant will lead to progressive loss of bone strength, which is highly undesirable. Eventually, this may lead to mechanical fatigue failure of the implant, as a result of ongoing repetitive loading in the absence of supporting surrounding bone, or failure of the implant-bone construct, through cutting-out of the implant in the presence of soft surrounding bone. Because of this, rigid load bearing implants are not suitable for prophylactic surgery to prevent fractures in the elderly.

An approach that has found some success is the reduction of the concentration of forces on the lateral aspect of the femur at the time of a fall by use of externally applied semi-rigid hip protectors.

Hip fracture risk as a result of a fall while wearing such protective devices appears to be greatly reduced. Patient compliance with instructions for use is a problem, as patients are often reluctant or unable to consistently wear the hip protectors.

### BRIEF SUMMARY OF THE INVENTION

The implant system according to the invention provides a method of decreasing fracture risk by reducing the concentration of forces on the lateral aspect of the greater trochanter of the femur at the time of a fall. This is achieved by increasing the contact area involved in the impact of a fall. This measure alone reduces peak pressures and peak stresses to the lateral aspect of the femur. In addition, the implants according to the invention allow absorption of energy, through the selection of implant material, such that elastic and/or plastic deformation can occur; or through energy dissipation to the soft tissues between the implant and the lateral aspect of the femur. The end result of the implant is a decrease in energy transfer to the proximal femur, and the spread of such energy over a greater volume of bone, with resultant decreased fracture risk. The implant is surgically implanted so that patients will not be left unprotected because of their failure to comply with instructions.

The implants may be coupled with an implant system for mechanical re-enforcement of bone, utilizing a load-sharing device, to minimize stress shielding over a long time frame. Alternatively, the implant system for mechanical re-enforcement of bone can be used on its own merits, decreasing fracture risk by strengthening the area of proximal femur.

The implants according to the invention provide enforcement of the area at risk for fracture, using implants designed to avoid excessive stress shielding of the surrounding bone. This is achieved through use of a compliant, flexible material so that the implant is similar in stiffness to the surrounding bone. In essence, the implant is only fully loaded at the time of excessive impact, when fracture could occur. By ensuring ongoing loading of the surrounding bone, progressive bone loss due to stress shielding can be minimized or avoided. In addition, a flexible implant is less likely to cut out of soft bone, when fully loaded.

The implants can be placed with minimally invasive surgery. Bone preparation through placement of drill holes, etc. is not necessary or is greatly reduced with the implants designed to purely decrease the peak pressures and stresses, and to minimize energy transfer. If fracture surgery is necessary, the presence of the prophylactic implant should not pose significant difficulties for the treating surgeon. If a fracture should occur with an implant in situ, the implant should have sufficient strength and stiffness to minimize the risk of displacement, disruption of the blood supply to the femoral head, and fracture bleeding. In this way, the likelihood is increased that the fracture can heal without formal fracture surgery, thus avoiding the significant risks associated with fracture surgery.

The implant system is for a femur, the femur having a femoral neck, a femoral head and a lateral femoral cortex, the system including an implant placeable inside the femoral neck through an opening in the lateral femoral cortex, and the implant extending from said opening to a positioned within the femoral head. The implant system may include a second implant starting from the opening to a second position within the femoral head; and a third implant, the third implant extending from the opening to a third position in the femoral head, the first, second and third implants forming a partial cone.

Alternatively, the second implant may extend from a second opening in the lateral femoral cortex to a second position within the femoral head. The implants may be straight or curved. The implants may be connected to a plate in the area of said greater trochanter.

An implant system for a femur may be provided, the implant approximately congruent to the lateral aspect of said femur. The implant may be placed adjacent to the femur, deep to the tendinous insertion of abductor muscles; or may be placed deep to the subcutaneous tissues and superficial to the fascia lata; or may be placed deep to the fascia lata and superficial to the tendinous insertion of abductor muscles. The implant system may include a first component connected to a second component, the first component positioned in a plane approximately parallel to said second component. The implant may have elastomeric qualities.

### BRIEF DESCRIPTION OF THE DRAWINGS

FIG. 1 is a frontal view of a proximal femur;

FIG. 2 is a lateral view thereof;

FIG. 3 is an alternative frontal view thereof, showing the femoral neck and head in greater detail;

FIG. 4 is a frontal view of representations of common hip fracture patterns;

FIG. 5 is a lateral view thereof;

FIG. 6 is a frontal view of a femur showing the soft tissue around the hip;

FIGS. 7 and 8 are, respectively, frontal and lateral views of an implant according to the invention placed centrally in the femoral neck;

FIGS. 9 and 10 are, respectively, frontal and lateral views an alternative placement of an implant according to the invention in the femoral neck;

FIGS. 11 and 12 are, respectively, frontal and lateral views of implants according to the invention placed in the femoral neck through multiple insertion openings;

FIGS. 13 and 14 are, respectively, frontal and lateral views of an alternative placement of implants according to the invention in the femoral neck through multiple insertion openings;

FIGS. 15 and 16 are, respectively, frontal and lateral views of implants according to the invention placed in the femoral neck, through a single opening;

FIGS. 17 and 18 are, respectively, frontal and lateral views of implants placed in the femoral neck, through a single opening, extending from the lateral cortex of the femur to well into the femoral head;

FIGS. 19 and 20 are, respectively, frontal and lateral views of a single straight implant according to the invention placed centrally in the femoral neck, extending from the lateral cortex of the femur to well into the femoral head;

FIGS. 21 and 22 are, respectively, frontal and lateral views of a single straight implant according to the invention placed in an alternative position within the femoral neck;

FIGS. 23 and 24 are, respectively, frontal and lateral views of multiple straight implants according to the invention placed in the femoral neck through multiple insertion openings;

FIGS. 25 and 26 are, respectively, frontal and lateral views of multiple straight implants according to the invention placed in the femoral neck, through a single opening, extending from the lateral cortex of the femur to within the femoral head;

FIGS. 27 and 28 are, respectively, frontal and lateral views of multiple straight implants according to the invention placed in the femoral neck through multiple insertion openings, extending from the lateral cortex of the femur to within the femoral head;

FIGS. 29 and 30 are, respectively, frontal and lateral views of multiple straight implants according to the invention placed centrally in the femoral neck, extending from the lateral cortex of the femur to within the femoral head;

FIGS. 31 and 32 are, respectively, frontal and lateral views of a single straight implant according to the invention placed centrally in the femoral neck, extending from the lateral cortex of the femur to within the femoral head;

FIGS. 33 and 34 are, respectively, frontal and lateral views thereof, in an alternative position;

FIGS. 35 and 36 are, respectively, frontal and lateral views of multiple straight implants according to the invention placed in the femoral neck through multiple insertion openings;

FIGS. 37 and 38 are, respectively, show frontal and lateral views of multiple straight implants placed in the femoral neck, through a single opening, extending from the lateral cortex of the femur to within the femoral head;

FIGS. 39 and 40 are, respectively, frontal and lateral views of multiple straight implants according to the invention placed in the femoral neck through multiple insertion openings;

FIGS. 41 and 42 are, respectively, frontal and lateral views of multiple straight implants according to the invention placed in the femoral neck, through a single opening;

FIGS. 43 and 44 are, respectively, frontal and later views of a single curved implant according to the invention with an insertion point just beyond the subtrochanteric region;

FIGS. 45 and 46 are, respectively, frontal and lateral views of a single curved implant according to the invention with an insertion point well beyond the subtrochanteric region;

FIGS. 47 and 48 are, respectively, frontal and lateral views of multiple curved implants according to the invention, inserted through multiple openings with insertion points close together;

FIGS. 49 and 50 are, respectively, frontal and lateral views of multiple curved implants according to the invention wherein the insertion points significantly separated;

FIGS. 51 and 52 are, respectively, frontal and lateral views of multiple curved implants according to the invention, having a single insertion point;

FIGS. 53 and 54 are, respectively, frontal and lateral views of a single curved implant according to the invention, having an insertion point just beyond the subtrochanteric region;

FIGS. 55 and 56 are, respectively, frontal and lateral views of a single curved implant according to the invention, having an insertion point beyond the subtrochanteric region;

FIGS. 57 and 58 are, respectively, frontal and lateral views of multiple curved implants according to the invention having insertion points close together;

FIGS. 59 and 60 are, respectively, frontal and lateral views of multiple curved implants according to the invention, having insertion points significantly separated;

FIGS. 61 and 62 are, respectively, frontal and lateral views of multiple curved implants according to the invention, having a single insertion point;

FIGS. 63 and 64 are, respectively, frontal and lateral views of a single curved implant according to the invention, having an insertion point just beyond subtrochanteric region;

FIGS. 65 and 66 are, respectively, frontal and lateral views of a single curved implant according to the invention, having an insertion point well beyond subtrochanteric region;

FIGS. 67 and 68 are, respectively, frontal and lateral views of multiple curved implants according to the invention, having insertion points close together;

FIGS. 69 and 70 are, respectively, frontal and lateral views of multiple curved implants according to the invention, wherein insertion points significantly separated;

FIGS. 71 and 72, are, respectively, frontal and lateral views of multiple curved implants according to the invention, inserted through a single opening;

FIG. 73 is a frontal view of an alternative embodiment of an implant according to the invention, wherein the implant is placed directly adjacent to the bone;

FIG. 74 is a frontal view of an implant according to the invention, placed superficial to the tendinous insertion of the abductor muscles/origin of the vastus lateralis muscle and deep to the fascia lata;

FIG. 75 is a frontal view of an implant according to the invention, wherein the implant is placed superficial to the fascia lata, deep into the subcutaneous tissues;

FIG. 76 is a frontal view of an implant according to the invention with a component deep to the abductor tendon/origin of vastus lateralis and a component superficial to abductor tendon/origin of vastus lateralis and deep to fascia lata, the components connected;

FIG. 77 is a frontal view of an implant according to the invention, wherein the implant has a component deep to abductor tendon/origin of vastus lateralis and a component superficial to the fascia lata, the components connected;

FIG. 78 is a frontal view of an implant according to the invention wherein the implant has a component superficial to abductor tendon/origin of vastus lateralis and deep to the fascia lata, and a component superficial to the fascia lata, the components connected;

FIG. 79 is a frontal view of an implant according to the invention wherein the implant has a component deep to the abductor tendon/origin of vastus lateralis, a component superficial to the abductor tendon/origin of vastus lateralis and deep to the fascia lata, and a component superficial to the fascia lata, the components connected;

FIGS. 80 and 81 are, respectively, frontal and lateral views of an implant according to the invention, wherein a single implant is placed into the femoral neck;

FIGS. 82 and 83, are, respectively, frontal and lateral views of an implant according to the invention wherein the long axis of the implant is parallel to the long axis of the femoral neck;

FIGS. 84 and 85 are, respectively, frontal and lateral views of multiple implants according to the invention placed into the femoral neck, with the long axis of the implants perpendicular to the long axis of the femur;

FIGS. 86 and 87 are, respectively, frontal and lateral views of multiple implants according to the invention wherein the long axis of the implants is parallel to the long axis of the femoral neck;

FIGS. 88 and 89 are, respectively, frontal and lateral views of multiple implants according to the invention, wherein the long axis of the cone is perpendicular to the long axis of the femur, in a plane coronal to the femoral neck, midway between its anterior and posterior border;

FIGS. 90 and 91 are, respectively, frontal and lateral views of multiple implants according to the invention wherein the long axis of the cone is parallel to the long axis of the femoral neck;

FIG. 92 is a frontal view of an implant according to the invention extending distally and intimately connected with the more distal femur;

FIG. 93 is a perspective view of a concave implant according to the invention; and

FIG. 94 is a perspective view of a raised rectangle implant according to the invention.

### DETAILED DESCRIPTION OF THE INVENTION

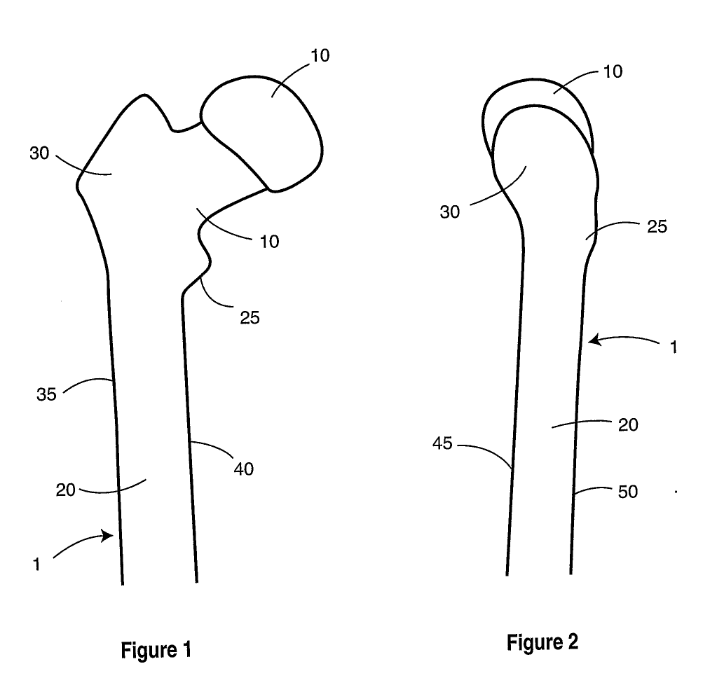

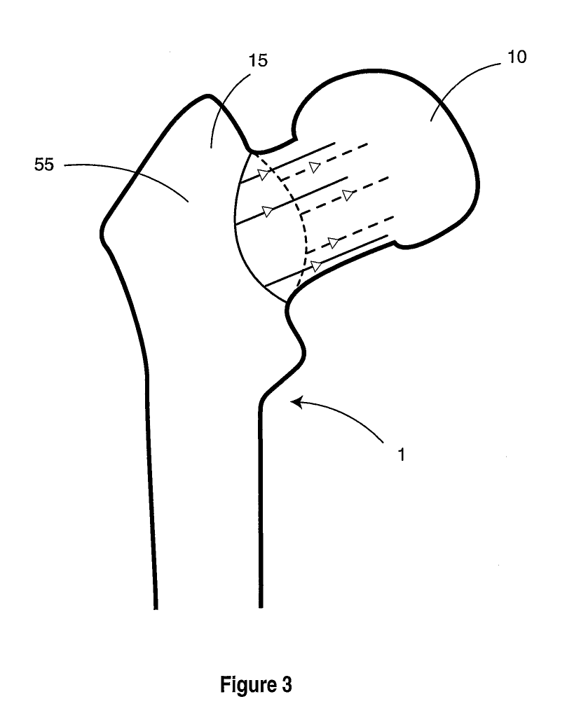

FIGS. 1 through 3 provide views of proximal femur 1. Components of the femur, as seen in FIGS. 1 through 3, include femoral head 10, femoral neck 15, femoral shaft 20, lesser trochanter 25, greater trochanter 30, medial femoral cortex 35, lateral femoral cortex 40, anterior femoral cortex 45, and posterior femoral cortex 50. Blood supply 55 runs from femoral neck 15 to femoral head 10.

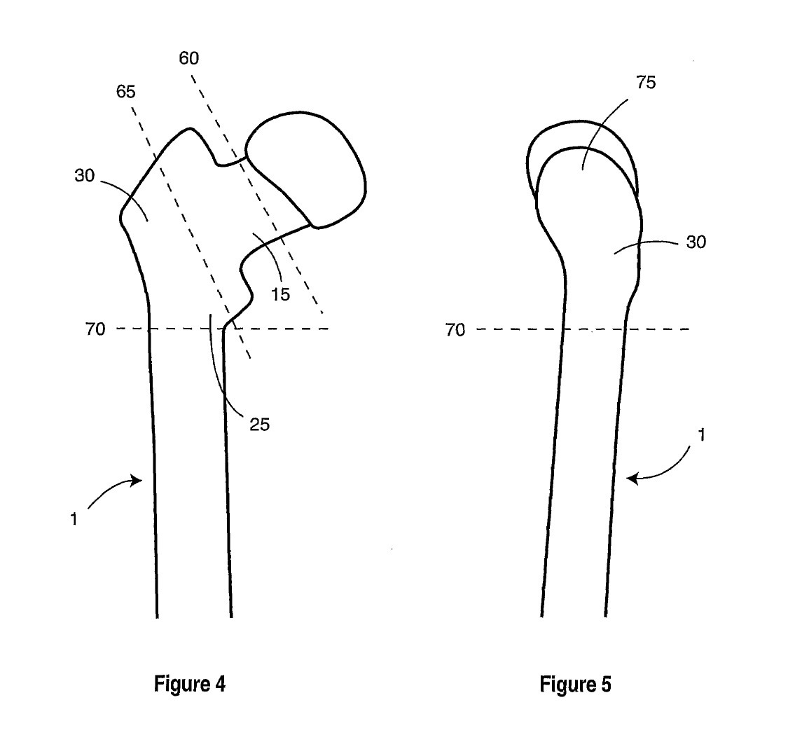

FIGS. 4 and 5 show typical fractures that may occur in the femur. Femoral neck fracture 60 occurs in femoral neck 15. Intertrochanteric fracture 65 occurs in the lesser trochanter 25 or greater trochanter 30. Subtrochanteric fracture 70 occurs below lesser trochanter 25. In a typical fall area 75 absorbs the direct impact.

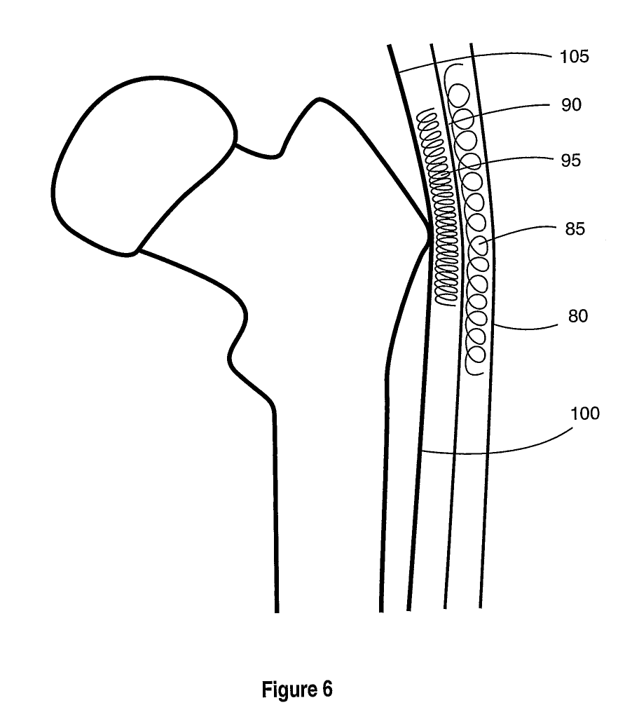

FIG. 6 shows the soft tissue surrounding the femur. Skin 80 forms an outer layer. Subcutaneous soft tissue 85 is beneath skin 80. Fascia lata 90 and bursa 95 are also within the soft tissue. The abductor insertion point 105 and point of origin of the vastus lateralis 100 are also present.

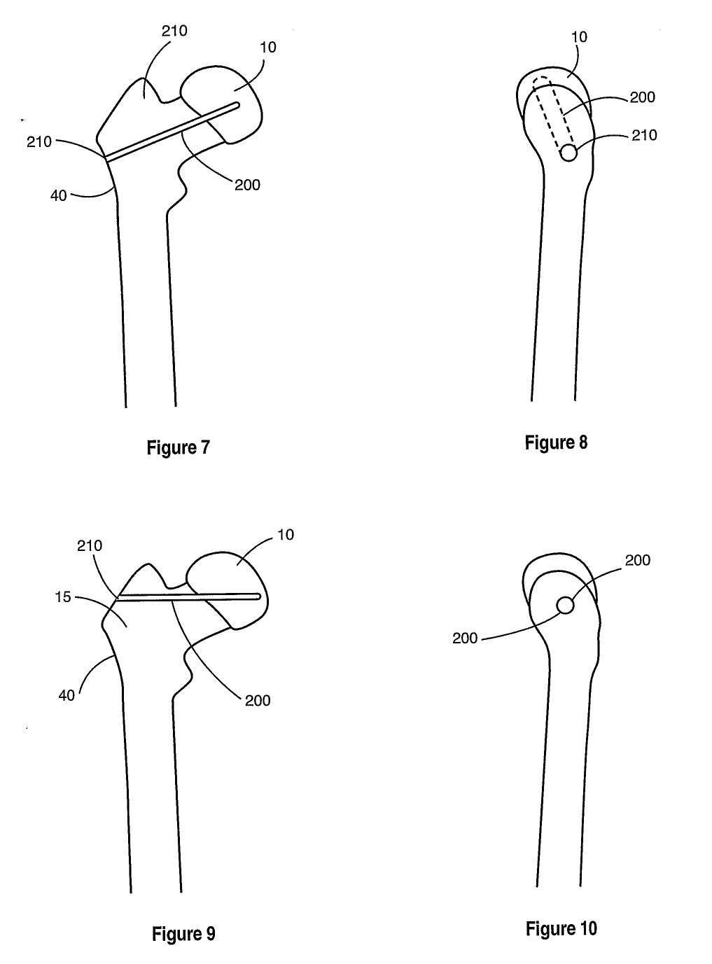

As seen in FIGS. 7 through 10, a single straight implant 200 according to the invention may be used.

As seen in FIGS. 7 and 8, implant 200 may be placed centrally in femoral neck 15, extending from the lateral femoral cortex 40 to within femoral head 10, with the long axis 210 of the implant approximately parallel to the long axis of femoral neck 15. In this position, implant 200 provides protection against femoral neck fractures 60 and intertrochanteric fractures 65. Dimensions of implant 200 vary with femoral neck 15 dimensions, quality of bone and implant 200 properties, such as strength and stiffness. When placed as a single implant 200, the diameter of implant 200 would typically be between 10-25 mm. This positioning of implant 200 is commonly used as part of a rigid, load bearing, metallic construct to treat fractures of the proximal femur.

As seen in FIGS. 9 and 10, implant 200 may be placed centrally in femoral neck 15, extending from the lateral femoral cortex 40 to within femoral head 10, with the long axis of implant 200 approximately perpendicular to the long axis of femoral shaft 20, in a plane coronal to femoral neck 15, approximately midway between the anterior and posterior aspects of femoral neck 15. This positioning of implant 200 provides protection against femoral neck fractures 60 and intertrochanteric fractures 65. Dimensions of implant 200 vary with femoral neck 15 dimensions, quality of bone, and implant properties, such as strength and stiffness. When placed as a single cylindrical implant 200, the diameter of implant 200 is typically between 10-25 mm. This positioning may be used as part of a rigid, load bearing, metallic construct to treat fractures of the proximal femur.

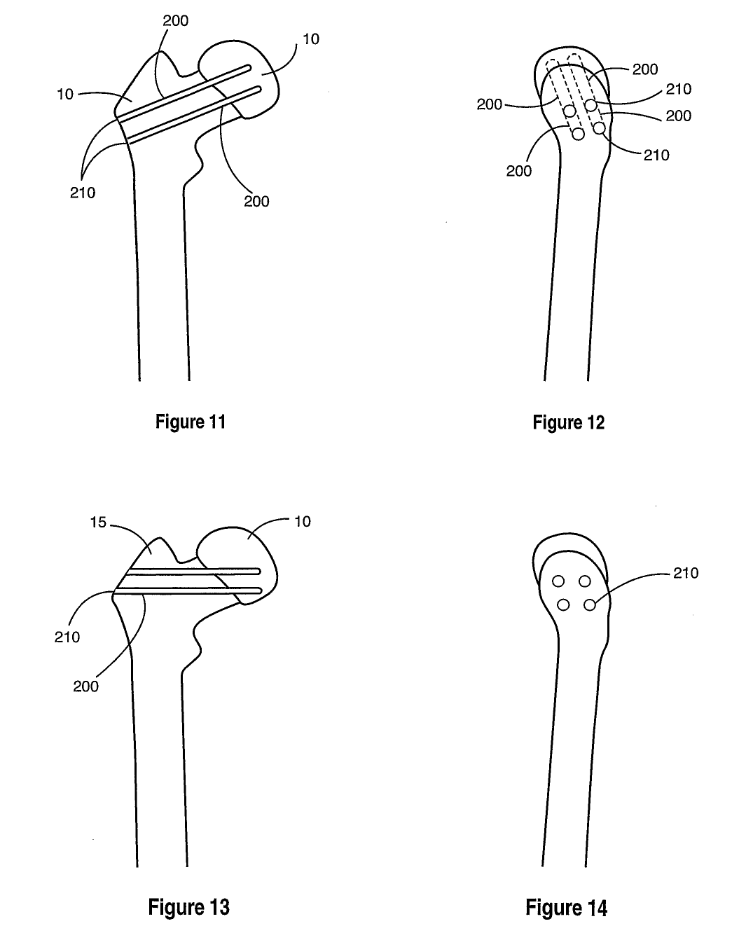

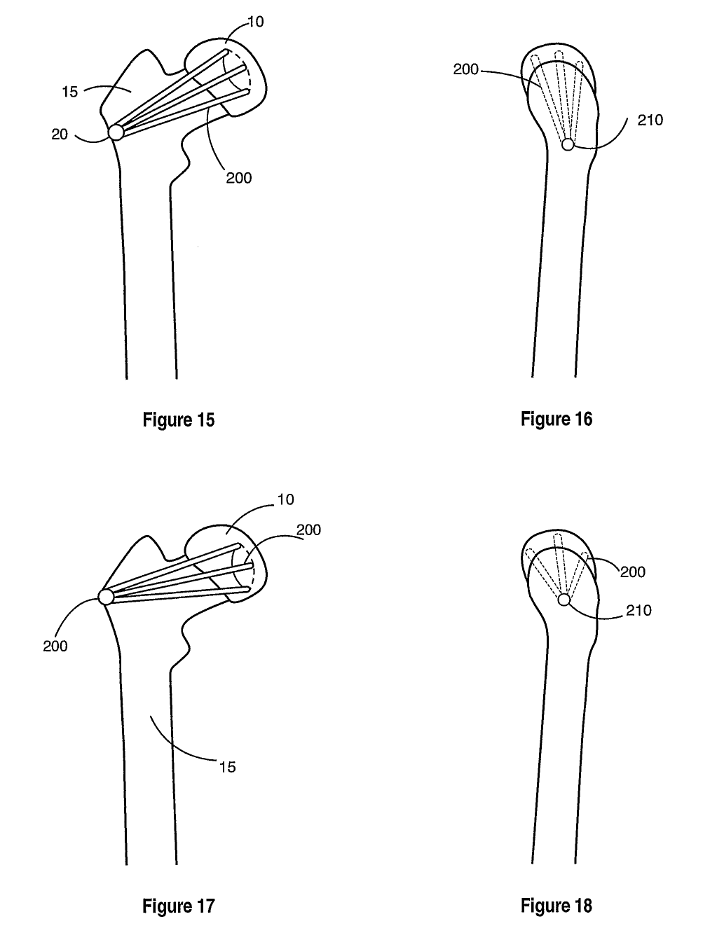

As seen in FIGS. 11 through 18, multiple straight implants 200 may be used. FIGS. 11 through 14 show multiple implants 200 inserted using multiple insertion openings 210 and FIGS. 15 through 1814 show multiple implants 200 inserted through a single insertion opening 210.

As seen in FIGS. 11 and 12, a plurality of implants 200 (four are shown in the Figures) may be placed in femoral neck 15 through multiple insertion openings 210, extending from the lateral femoral cortex 40 to within femoral head 10, with the long axis of implants 200 approximately parallel to the long axis of femoral neck 15. Implants 200 are spaced around the longitudinal center of femoral neck 15. This positioning of implants 200 provides protection against femoral neck fractures 60 and intertrochanteric fractures 65. The number of implants 200 used may vary with implant diameter, e.g. three or four implants 200 would be used having implant diameters of 6-8 mm. This positioning may be used as part of a rigid, load bearing, metallic construct to treat fractures of the proximal femur.

As seen in FIGS. 13 and 14, implants 200 may be placed in femoral neck 15 through multiple insertion openings 210, extending from the lateral femoral cortex 40 to within femoral head 10, with the long axis of implants 200 approximately perpendicular to the long axis of femoral shaft 20. Implants 200 are spaced around a line perpendicular to the long axis of femoral shaft 20, in a plane coronal to femoral neck 15, approximately midway between the anterior and posterior border of femoral neck 15. This positioning of implants 200 provides protection against femoral neck fractures 60 and intertrochanteric fractures 65. The number of implants 200 placed may vary with implant diameter, e.g. three or four implants 200 would be used if they had a diameter of 6-8 mm.

As seen in FIGS. 15 and 16, implants 200 may be positioned in femoral neck 15, through a single opening 210, each implant 200 extending from lateral femoral cortex 40 to within femoral head 10. Implants 200 will form a partial cone, with an apex at opening 210 in lateral femoral cortex 40 and the longitudinal axis of the cone approximately along the long axis of femoral neck 15. Implants 200 are spaced around the longitudinal axis of the cone. This positioning of implants 200 provides protection against femoral neck fractures 60 and intertrochanteric fractures 65. The number of implants 200 placed will vary with implant diameter, e.g. three or four implants 200 could be used in the case of implant diameters of 6-8 mm.

As seen in FIGS. 17 and 18, implants 200 may be placed in femoral neck 15, through a single opening 210, extending from the lateral femoral cortex 40 to within femoral head 10. Implants 200 will form a partial cone, with an apex at opening 210 and the longitudinal axis of the cone approximately perpendicular to the long axis of femoral shaft 20; in a plane coronal to femoral neck 15, and midway between the anterior and posterior border of femoral neck 15. Implants 200 are spaced around the longitudinal axis of the cone. This positioning of implants 200 provides protection against femoral neck fractures 60 and intertrochanteric fractures 65. The number of implants 200 placed will vary with implant diameter, e.g. three or four implants would be used in the case of implant diameter of 6-8 mm.

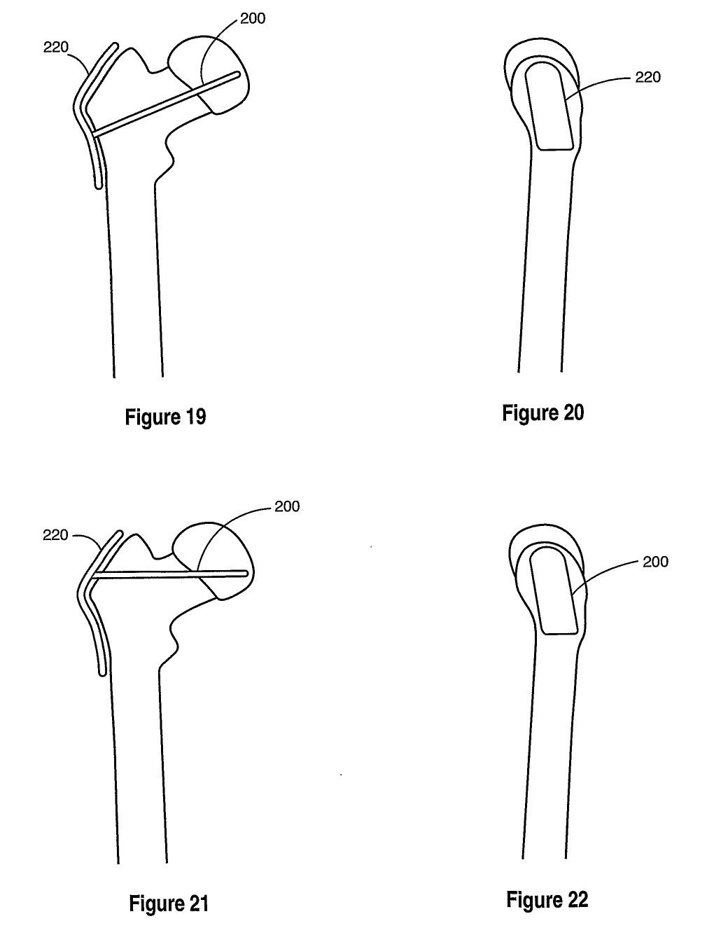

As seen in the FIGS. 19 through 30, implant(s) 200 may be straight and connected to a side plate in the region of greater trochanter 30.

As seen in FIGS. 19 and 20, a single straight implant 200 may be placed centrally in femoral neck 15, extending from lateral femoral cortex 40 to within femoral head 10, with the long axis of implant 200 approximately parallel to the long axis of femoral neck 15. Implant 200 is connected to plate 220 in the area of greater trochanter 30. Plate 220 is generally flat and shaped to cover a portion of femur 1. This positioning of implant 200 provides protection against femoral neck fractures 60 and intertrochanteric fractures 65 through reinforcement of femoral neck 15 and the intertrochanteric area, as well as through providing a larger surface area at the time of impact, reducing the peak pressure at the level of the most lateral aspect of greater trochanter 30, as well as through energy absorption, such as elastic or plastic deformation of plate 220, reducing the energy transfer to femoral neck 15 and intertrochanteric area. This positioning may be used as part of a rigid, load bearing, metallic construct to treat fractures of proximal femur 1.

As seen in FIGS. 21 and 22, a single straight implant 200 may be placed centrally in femoral neck 15, extending from the lateral femoral cortex 40 to within femoral head 10, with the long axis of implant 200 approximately perpendicular to the long axis of femoral shaft 20; in a plane coronal to femoral neck 15. Approximately midway between the anterior and posterior aspect of femoral neck 15, implant 200 is connected to plate 220 in the area of greater trochanter 30. This positioning provides protection against femoral neck fractures and intertrochanteric fractures as described above with respect to FIGS. 19 and 20. This positioning may be used as part of a rigid, load bearing, metallic construct to treat fractures of proximal femur 1.

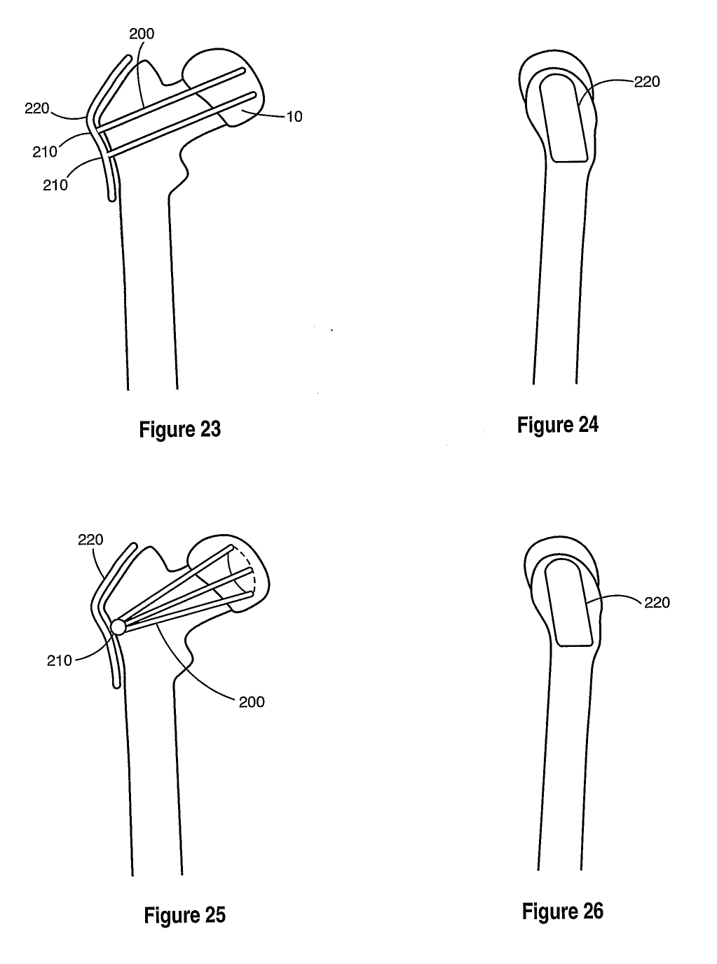

As seen in FIGS. 23 and 24, multiple straight implants 200 may be placed in femoral neck 15 through multiple insertion openings 210, extending from lateral femoral cortex 40 to within femoral head 10, with the long axis of implants 200 approximately parallel to the long axis of femoral neck 15. Implants 200 are spaced around the longitudinal center of femoral neck 15, and implants 200 are connected to plate 220 in the area of greater trochanter 30. This positioning of implants 200 and plate 220 provides protection against femoral neck fractures and intertrochanteric fractures as described above in relation to FIGS. 19 and 20.

As seen in FIGS. 25 and 26 multiple straight implants 200 may be placed in femoral neck 15, through a single opening 210, extending from lateral femoral cortex 40 to within femoral head 10. Implants 200 form part of a cone, with the apex of the cone at opening 210 in the lateral femoral cortex 40 and the longitudinal axis of the cone approximate to the long axis of femoral neck 15. Implants 200 are spaced around the longitudinal axis of the partial cone, and implants 200 are connected to plate 220 in the region of greater trochanter 30. This positioning provides protection against femoral neck fractures 60 and intertrochanteric fractures 65 as described above in relation to FIGS. 19 and 20.

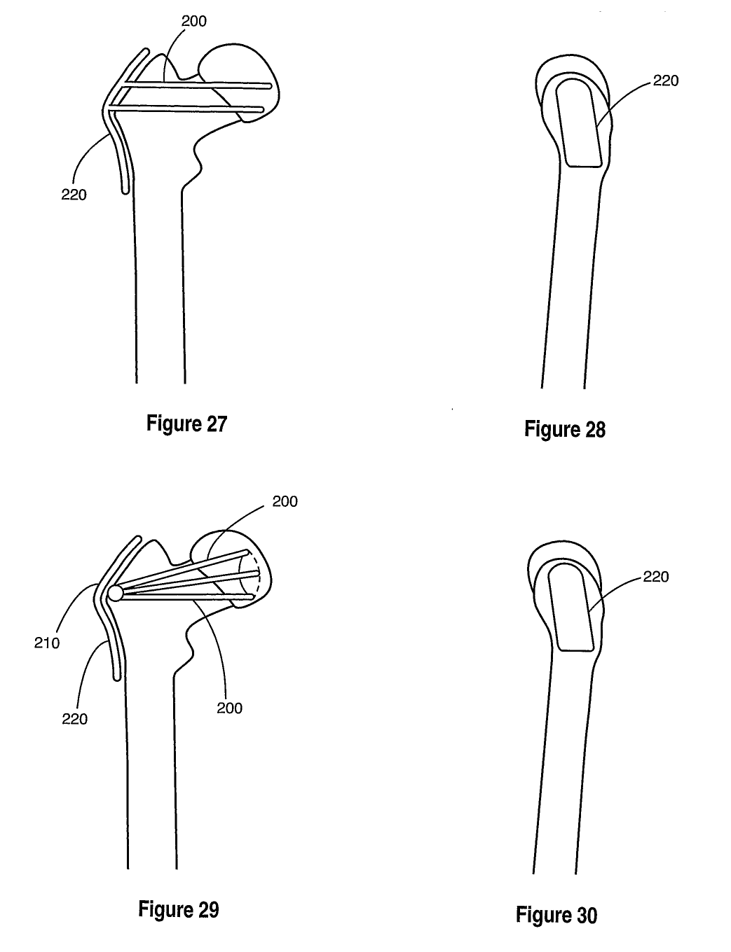

As seen in FIGS. 27 and 28, multiple straight implants 200 may be placed in femoral neck 15 through multiple insertion openings 210, extending from the lateral femoral cortex 40 to within femoral head 10, with the long axis of implants 200 approximately perpendicular to the long axis of femoral shaft 20. In a plane coronal to the femoral neck, approximately midway between the anterior and posterior aspect of femoral neck 15, implants 200 are connected to plate 220 in the area of greater trochanter 30. This positioning of implants 200 and plate 220 provides protection against femoral neck fractures 60 and intertrochanteric fractures 65 as described above in relation to FIGS. 19 and 20.

As seen in FIGS. 29 and 30, multiple straight implants 200 may be placed centrally in femoral neck 15, extending from lateral femoral cortex 40 to within femoral head 10. Implants 200 form part of a cone, with the apex of the cone at opening 210 in lateral femoral cortex 40 and the longitudinal axis of the cone approximately perpendicular to the long axis of femoral shaft 20. In a plane coronal to femoral neck 15, approximately midway between the anterior and posterior aspect of femoral neck 15, implants 200 are connected to plate 220 in the area of greater trochanter 30. This positioning of implants 200 and plate 220 provides protection against femoral neck fractures and intertrochanteric fractures as described above in relation to FIGS. 19 and 20.

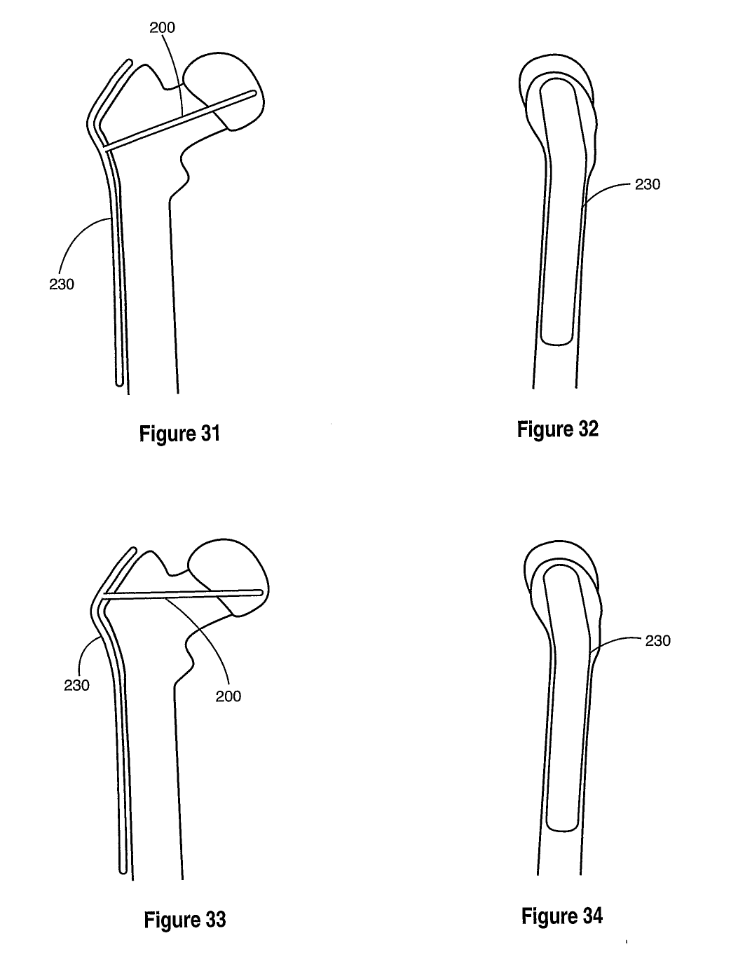

As seen in FIGS. 31 to 42, straight implant(s) 200 may be connected to a side plate 230 extending from the region of the greater trochanter distally to the subtrochanteric area or beyond.

As seen in FIGS. 31 and 32 single straight implant 200 may be placed centrally in femoral neck 15, extending from the lateral femoral cortex 40 to within femoral head 10, with the long axis of implant 200 approximately parallel to the long axis of femoral neck 15. Implant 200 is connected to plate 230 extending from the area of greater trochanter 30 to the subtrochanteric area and beyond. This positioning provides protection against femoral neck fractures 60, intertrochanteric fractures 65 and subtrochanteric fractures 70 through reinforcement of femoral neck 15, and the intertrochanteric and subtrochanteric areas, as well as providing a larger surface area at the time of impact, reducing the peak pressure at the level of the most lateral aspect of greater trochanter 30, as well as possibly through energy absorption, such as elastic or plastic deformation of plate 230, reducing the energy transfer to femoral neck 15, and the intertrochanteric and subtrochanteric areas.

As seen in FIGS. 33 and 34, single straight implant 200 may be placed centrally in femoral neck 15, extending from lateral femoral cortex 40 to within femoral head 10, with the long axis of implant 200 approximately perpendicular to the long axis of femoral shaft 20. In a plane coronal to femoral neck 15, approximately midway between the anterior and posterior aspect of femoral neck 15, implant 200 is connected to plate 230 in the area of greater trochanter 30 extending from the area of greater trochanter 30 to the subtrochanteric area and beyond. This positioning of implant 200 and plate 230 provides protection against femoral neck fractures 60, intertrochanteric fractures 65 and subtrochanteric fractures 70 as described above in relation to FIGS. 31 and 32.

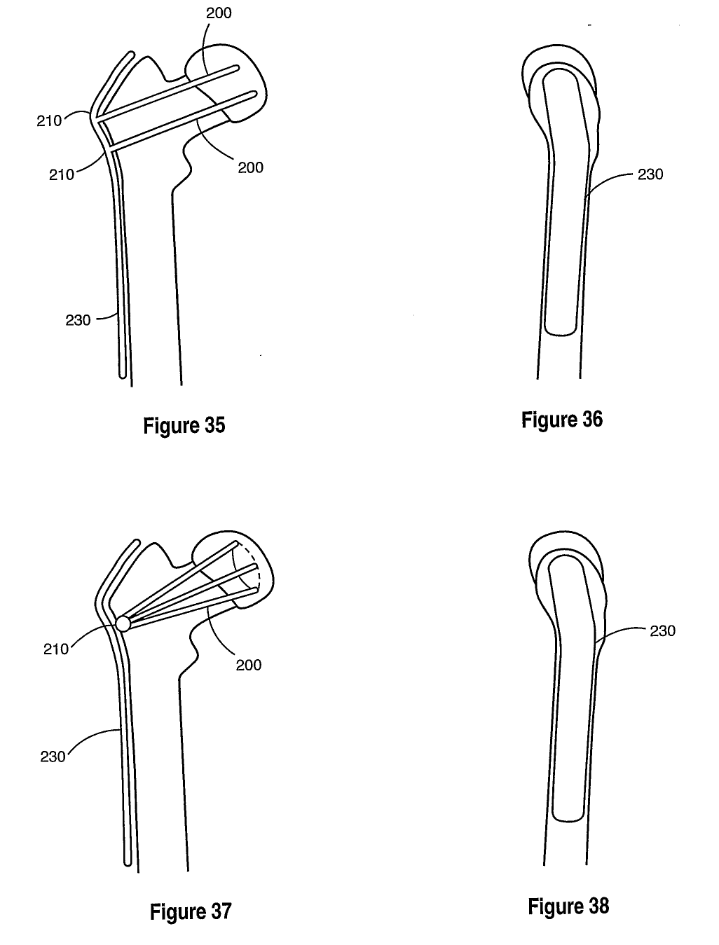

As seen in FIGS. 35 and 36, multiple straight implants 200 may be placed in femoral neck 15 through multiple insertion openings 210, extending from lateral femoral cortex 40 to within femoral head 10, with the long axis of implants 200 approximately parallel to the long axis of femoral neck 15. Implants 200 are spaced around the longitudinal center of femoral neck 15, and implants 200 are connected to plate 230 in the area of greater trochanter 30, plate 230 extending from the area of greater trochanter 230 to the subtrochanteric area and beyond. This positioning of implants 200 and plate 230 provides protection against femoral neck fractures 60, intertrochanteric fractures 65 and subtrochanteric fractures 70 as described in relation to FIGS. 31 and 32.

As seen in FIGS. 37 and 38, a plurality of straight implants 200 may be placed in femoral neck 15, through a single opening 210, extending from the lateral femoral cortex 40 to within femoral head 10. Implants 200 form a partial cone, having an apex at opening 210 in lateral femoral cortex 40 and a longitudinal axis approximate to the long axis of femoral neck 15. Implants 200 are spaced around the longitudinal axis of the cone, and implants 200 are connected to plate 230 extending from the area of greater trochanter 30 to the subtrochanteric area and beyond. This positioning of implants 200 and plate 230 provides protection against femoral neck fractures 60, intertrochanteric fractures 65 and subtrochanteric fractures 70 as described in relation to FIGS. 31 and 32.

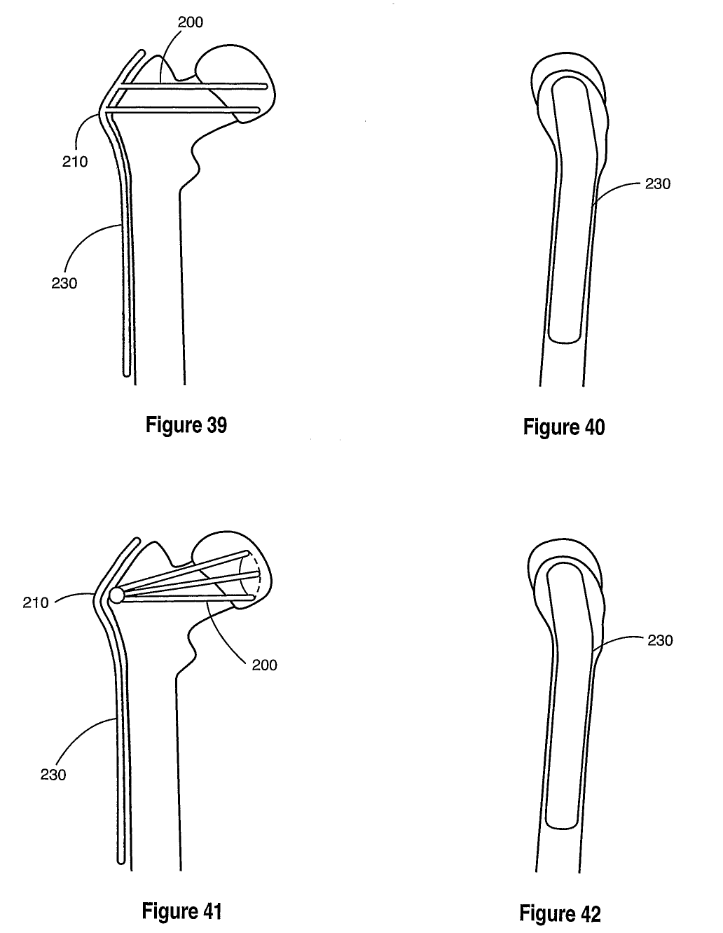

As seen in FIGS. 39 and 40, a plurality of straight implants 200 may be placed in femoral neck 15 through multiple insertion openings 210, extending from lateral femoral cortex 40 to within femoral head 10, with the long axis of implants 200 approximately perpendicular to the long axis of femoral shaft 20. In a plane coronal to femoral neck 15, approximately midway between the anterior and posterior aspect of the femoral neck 15, implants 200 are connected to plate 230 extending from the area of greater trochanter 30 to the subtrochanteric area and beyond. This positioning of implants 200 and plate 230 provides protection against femoral neck fractures, intertrochanteric fractures and subtrochanteric fractures as described in relation to FIGS. 31 and 32.

As seen in FIGS. 41 and 42, a plurality of straight implants 200 may be placed in femoral neck 15, through a single opening 210, extending from lateral femoral cortex 40 to within femoral head 10. Implants 200 form part of a cone, with the apex of the cone at the entry opening 200 in the lateral femoral cortex 40 and the longitudinal axis of the cone approximately perpendicular to the long axis of femoral shaft 20. In a plane coronal to femoral neck 15, approximately midway between the anterior and posterior aspect of femoral neck 15, implants 200 are connected to plate 230 extending from the area of greater trochanter 30 to the subtrochanteric area and beyond. This positioning of implants 200 and plate 230 provide protection against femoral neck fractures 60, intertrochanteric fractures 65 and subtrochanteric fractures 70 as described in relation to FIGS. 31 and 32.

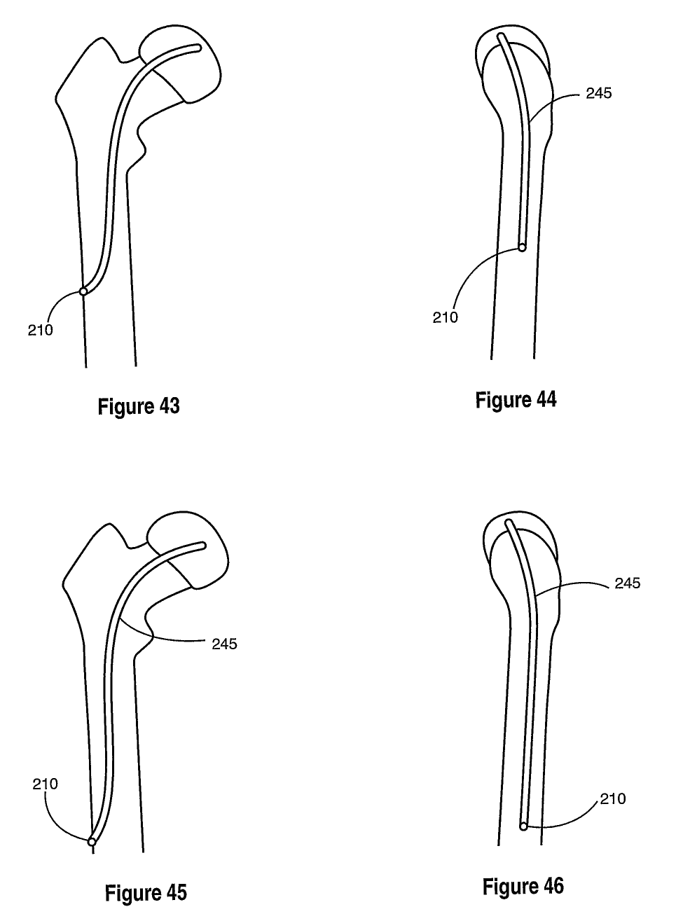

As seen in FIGS. 43 to 72, implants 240 may be curved. These implants are inserted through the lateral femoral cortex 40, distally to the subtrochanteric region, thus extending the area of protection against fracture to the subtrochanteric area and beyond. Flexible metallic implants may be used for fixation of fractures of proximal femur 1, to provide semi-rigid fixation, sufficiently stable to allow bone healing.

As seen in FIGS. 43 to 46 a single curved implant 240 may be used. FIGS. 43 and 44 show insertion point 210 just beyond the subtrochanteric region and FIGS. 45 and 46 show an insertion point 210 well beyond the subtrochanteric region, allowing and the use of an elongated curved implant 245, and extending the area of protection against fracture.

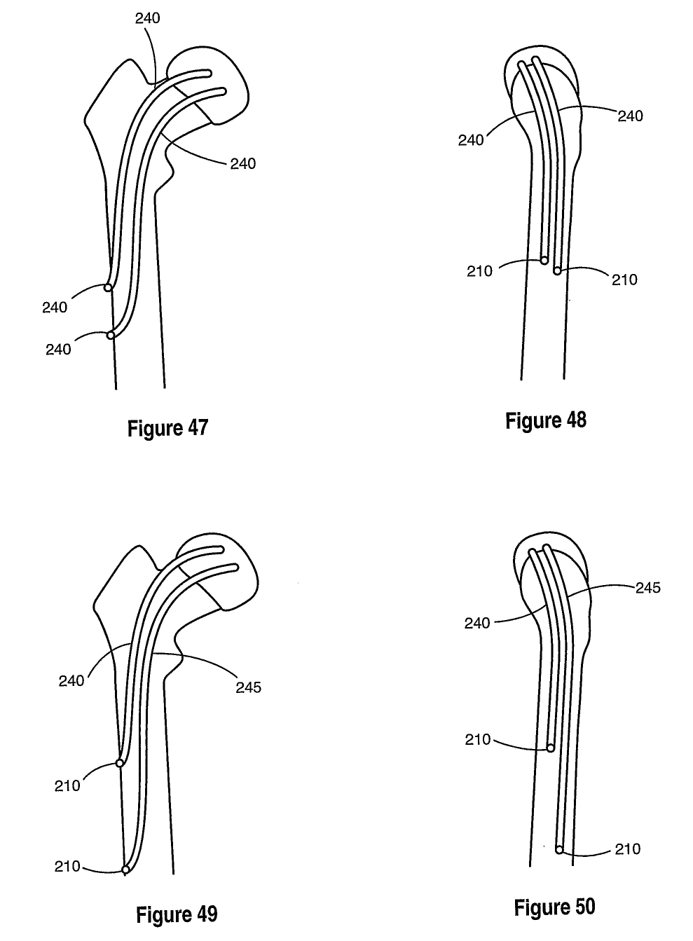

As seen in FIGS. 47 to 50 multiple curved implants may be used, inserted through multiple openings 210. FIGS. 47 and 48 show insertion points 210 in close proximity and FIGS. 49 and 50 show insertion points 210 significantly separated, to provide variable protection for portions of the femur.

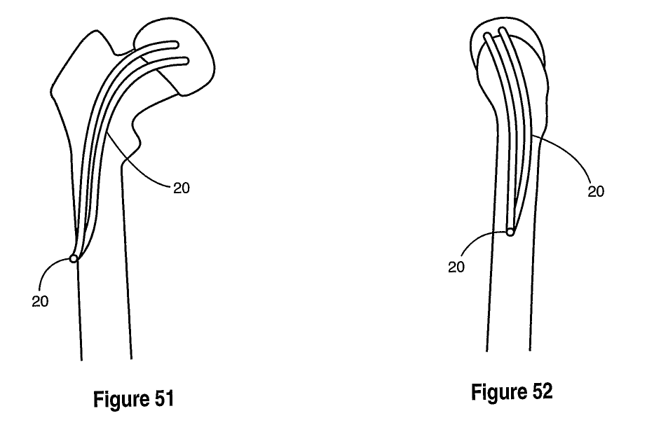

As seen in FIGS. 51 and 52, a plurality of curved implants 240 may be used, inserted through a single opening 210.

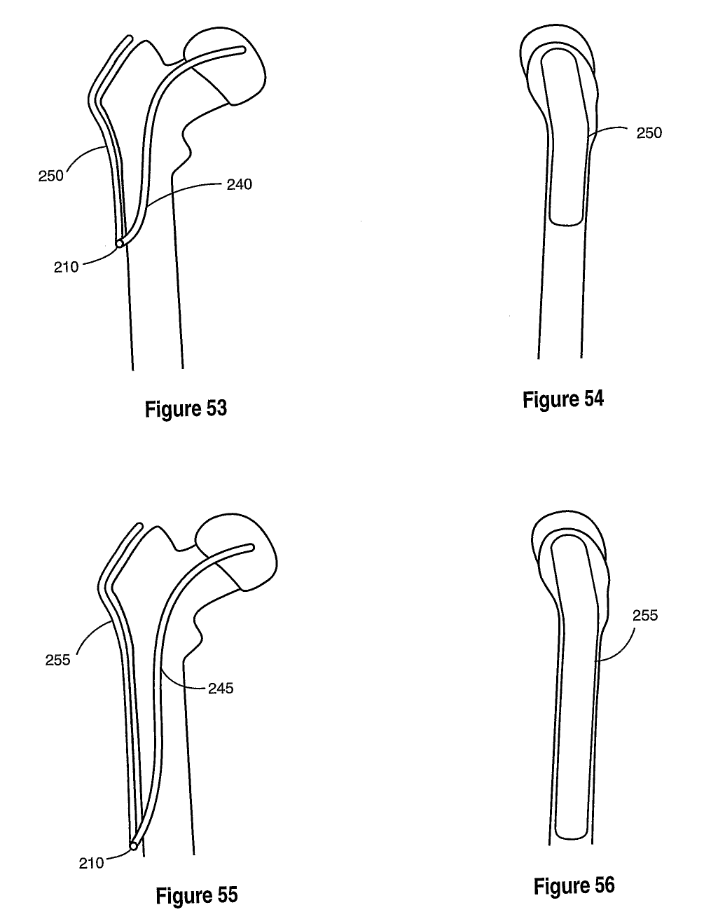

As seen in FIGS. 53 to 56, a single curved implant 240 or elongated implant 245 may be used, attached to a plate 250 or elongated plate 255, respectively, extending from the area of greater trochanter 30 to the subtrochanteric region and beyond, to include the point of insertion 210 of the implant 240 or elongated implant 245. FIGS. 53 and 54 show an insertion point 210 just beyond the subtrochanteric region and FIGS. 55 and 56 show an insertion point 210 well beyond the subtrochanteric region, extending the area of protection against fracture.

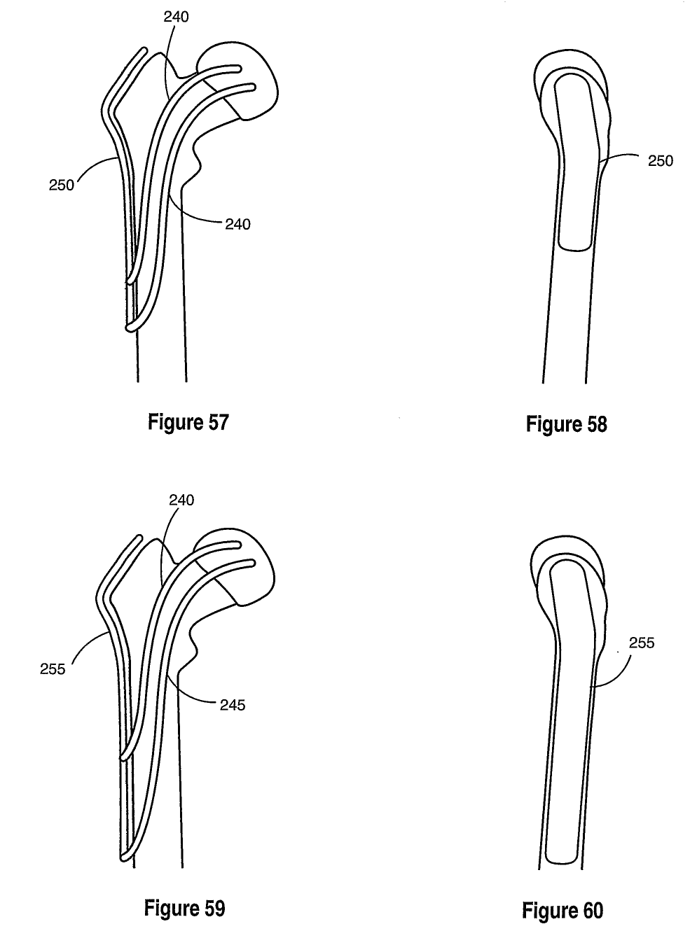

As seen in FIGS. 57 to 60, multiple curved implants 240 or elongated implants 245 may be used, inserted through multiple openings 210, attached to a plate 250 or 255 extending from the area of greater trochanter 30 to the subtrochanteric area and beyond, to include the point of insertion 210 of implants 240 or 245. FIGS. 57 and 58 show insertion points 210 in close proximity and FIGS. 59 and 60 show insertion points 210 significantly separated, to provide variable protection for various portions of the femur.

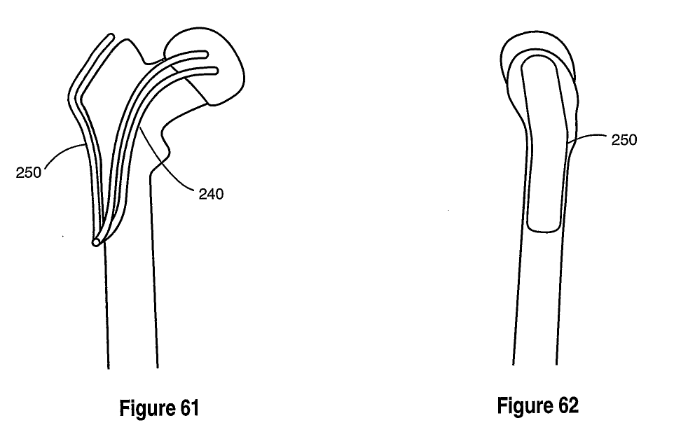

As seen in FIGS. 61 and 62, a plurality of curved implants 240 may be used, inserted through a single opening 210, attached to a plate 250 extending from the area of greater trochanter 30 to the subtrochanteric area and beyond, to include the point of insertion 210 of the implants 240.

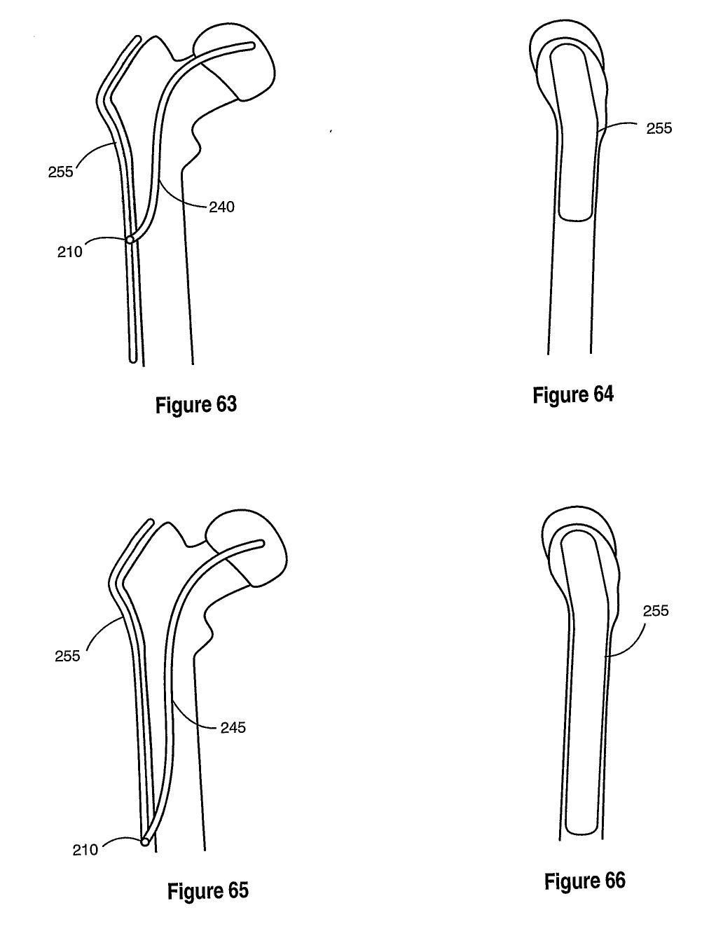

As seen in FIGS. 63 to 66, a single curved implant 240 or 245 may be used, attached to a plate 250 or 255 extending from the area of greater trochanter 30 to the subtrochanteric area and beyond, and extending distally beyond the point of insertion 210 of the implants 240 or 245, to further re-enforce the femoral shaft 20. FIGS. 63 and 64 show an insertion point 210 just beyond the subtrochanteric region and FIGS. 65 and 66 show an insertion point 210 well beyond subtrochanteric region, extending the area of protection against fracture

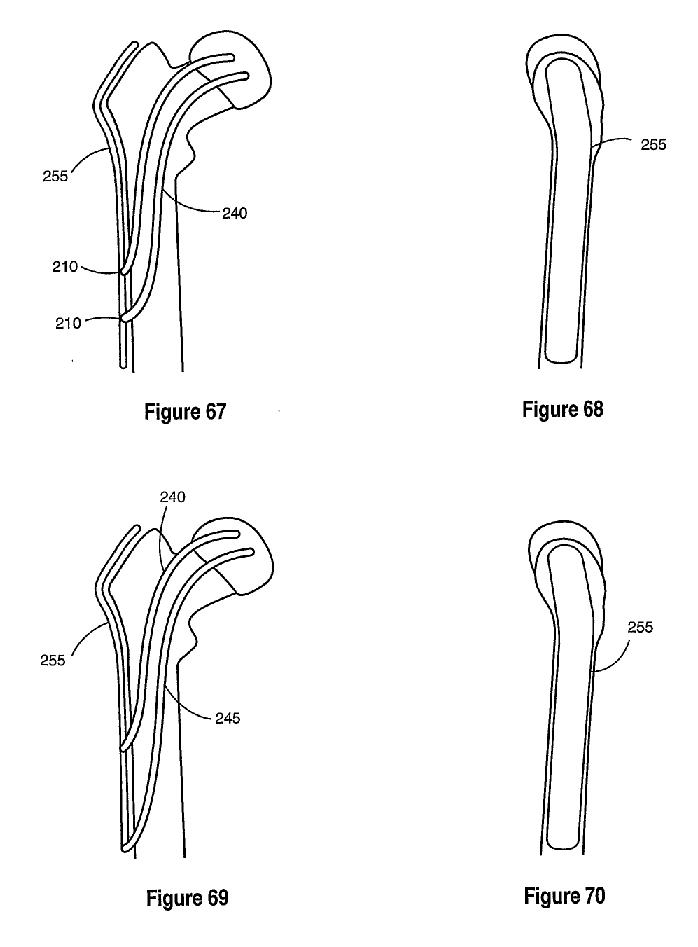

As seen in FIGS. 67 to 70, a plurality of curved implants 240 or 245 may be used, inserted through multiple openings 210, attached to elongated plate 255 extending from the area of greater trochanter 30 to the subtrochanteric area and beyond, to extend distally beyond the point of insertion 210 of the implants, to further re-enforce femoral shaft 20. FIGS. 67 and 68 show the insertion points 210 in close proximity and FIGS. 69 and 70 show the insertion points 210 significantly separated, to provide variable protection against various portions of the femur.

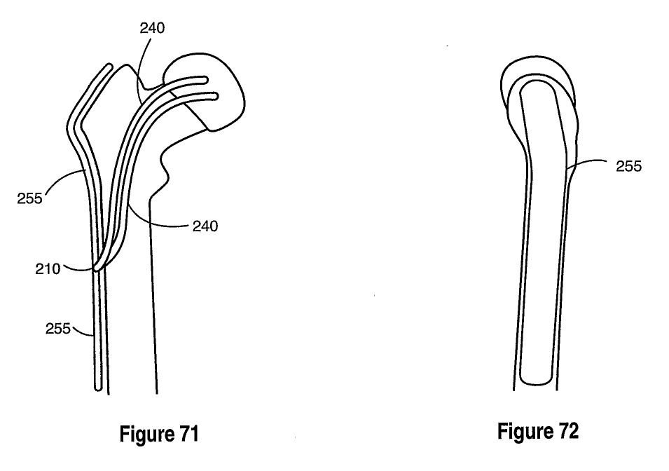

As seen in FIGS. 71 and 72, a plurality of curved implants 240 may be used, inserted through a single opening 210, attached to plate 255 extending from the area of the greater trochanter 30 to the subtrochanteric area and beyond, to extend distally beyond the point of insertion 210 of the implants 240, to further re-enforce the femoral shaft 20.

A single implant may be placed inside femoral neck 15 through an opening 210 created in the lateral femoral cortex 40 of the femur 1, extending from the lateral femoral cortex 40 to within femoral head 10. Such an implant has sufficient strength to withstand forces associated with low and moderate energy falls. This implant is sufficiently compliant for it to act as a load sharing device, thereby facilitating ongoing loading of the surrounding bone, sufficiently to minimize stress shielding of the bone. This increases the overall strength of the region of the proximal femur 1 over a sustained period of time.

Alternatively, a plurality of implants of smaller diameter may be placed within the femoral neck 15, through one or more smaller bore openings 210 created in the lateral femoral cortex 40, extending from the lateral cortex of the femur to within femoral head 10. The aggregate of implants 200 or 245 has sufficient strength to withstand forces associated with low and moderate energy falls. This aggregate of implants is sufficiently compliant for it to act as a load sharing device, thereby facilitating ongoing loading of the surrounding bone, sufficiently to minimize stress shielding of the bone. This increases the overall strength of the region of the proximal femur 1 over a sustained period of time.

The implant may alternatively be connected to a plate just lateral to the femoral cortex in the area of the greater trochanter 30, which provides an increased surface for distribution of the forces impacting the region of the greater trochanter 30 during a fall. Such a plate may have the ability to absorb energy, thus enhancing dissipation of energy during a fall prior to energy transmission to the proximal femur 1, leading to diminished fracture risk.

The implants may be connected to each other and to a plate just lateral to the femoral cortex in the area of the greater trochanter 30, thereby increasing the strength of the construct, as well as providing an increased surface for distribution of the forces impacting the region of the greater trochanter 30 during a fall. Such a plate may have the ability to absorb energy, thus enhancing dissipation of energy during a fall prior to energy transmission to the proximal femur 1, leading to diminished fracture risk.

The long axis of the implants may be straight. Alternatively, the long axis of the implants may be curved. The curved nature of the implants permits extension of the area of reinforcement of the femur through intramedullary implant placement to the subtrochanteric area and beyond

The implants may be connected to a plate, which extends distally and is intimately connected with the more distal femur, thus extending the area of reinforcement to the subtrochanteric area and beyond. This plate may be sufficiently compliant for the plate to act as a loadsharing device, thereby allowing ongoing loading of the surrounding bone, sufficiently to minimize stress shielding of the bone. This increases the overall strength of the region of the proximal femur 1 through intramedullary implant placement and of the more distal femur, as covered by the implant, through extra-medullary implant placement, over a sustained period of time.

The implant(s) may have a solid core. Alternatively, the core of the implant may be hollow. The cross-section of the implants may be a circle (or part thereof), ellipse (or part thereof), polygon (or part thereof), poly-foil or cloverleaf or otherwise flanged (or part thereof), curved or a straight line segment. The cross-section of an implant with a hollow-core may be open or closed.

The implants may be made, at least partially, out of a non-porous metal alloy as commonly used in orthopaedic surgery, such as, but not limited to, cobalt chrome alloy, stainless steel, titanium alloy. The implants may also be made, at least partially, out of porous metals, such as, but not limited to, trabecular or porous tantalum, and trabecular or porous alloys, such as certain titanium-nickel formulations.

Furthermore, the implants may be are made, at least partially, out of synthetic materials such as, but not limited to, polyethylene of varying molecular weight and varying degree of cross-linking, polyurethane of varying composition, poly-aryl-ether-ketone and poly-ether-ether-ketone of varying composition, and/or polymethylmethylacrylate. The implants may be formed and provided with final shape, strength and stiffness prior to implantation, or they may form and achieve final shape, strength and stiffness after implantation. The latter is particularly applicable to selfcuring materials, either cold curing, such as, but not limited to, certain polyurethanes, or hot curing (exothermic reaction), such as polymethylmethacrylate. Certain thermoplastics may allow final shape adjustments to be made upon application of external heat to the implant, at the time of implantation.

The implants may also be made, at least partially, out of a mineral substrate such as, but not limited to, coral, hydroxy-apatite, calcium phosphate formulations, human bone or derivatives (autogenous or allogenous, either directly harvested and subsequently processed or a result of bio-engineering processes and in-vivo or in-vitro tissue culture, including, but not limited to, the use of stem cells), animal bone or derivatives, synthetic bone or derivatives, zirconia and alumina ceramics as commonly used in orthopaedic surgery, porous and/or polycrystalline silicon.

The implants may be made out of a composite of materials, such as, but not limited to, those described above.

The implant may be secured in position, at least partially, through threading of at least part of the surface of the implant. The implant may also be secured in position, at least partially, through a press-fit arising from a mismatch between the geometry of implant and the space prepared to accept the implant.

The implant may also be secured in position, at least partially, through a relatively thin layer of appropriately formulated polymethylmethacrylate (commonly known as ‘bone cement’), not unlike the fixation of cemented joint replacement components. Furthermore, the implant may be secured in position, at least partially, by bone or soft tissue ingrowth and/or ongrowth, as may be induced or conducted by the characteristics of the surface of the implant, such as, but not limited to, the presence of pores, beads, crevices, coating such as plasma spray, hydroxyl apatite or calcium phosphate formulations, the presence of biologically active agents, such as bone or soft tissue metabolism modulating agents, vectors or inductors for gene therapy, enzymes or catalysts.

The implant may also be secured in position, at least partially, by implant geometry that resists motion into the direction opposite to insertion, such as, but not limited to, the following: the presence of flexible barbs; or implant geometry with the diameter of part of the implant greater than the residual diameter of the opening created in the femoral cortex for introduction of the implant, facilitated by introduction of the implant in liquid or viscous form, such as applicable to certain self-curing synthetic materials as described above, use of sufficient force in the direction of the long axis of the implant to induce an elastic response in the implant, leading to a transient decrease in its diameter, sufficient to allow implant introduction, followed by expansion to, or close to, the initial pre-insertion diameter; or use of a closing technique after insertion of the implant into the femoral neck, such as, but not limited to the use of a plug (autologous or allogeneic bone or soft tissue, solid or porous metal, synthetic material, ceramic material, secured in place through press-fit and secondary biological fixation or with the aid of a grout or adhesive, e.g. polymethylmethacrylate).

The implants may decrease the risk of hip fracture after a fall not only through mechanical reinforcement of a femoral segment, but also through modulation of local metabolism, such as, but not limited to, the metabolism of bone, blood vessels, connective tissue, and the immune system, through acting as a carrier for biologically active material, such as vectors and inductors for gene therapy, stem cells or stem cell derived therapeutic entities, biological factors directly involved in bone metabolism, pharmacological agents and modulating agents, or by displaying a structure conductive and/or inductive to bone formation.

The implants should be sufficiently strong, rigid and well fixed after insertion to minimize, in case of a hip fracture after a low to moderate energy fall, the risk of fracture fragment displacement, disruption to the blood supply of the bone and the amount of fracture bleeding, thus increasing the probability that the fracture is stable, with adequate blood supply to the femoral head, which will facilitate fracture healing without formal fracture surgery. Minimizing the amount of fracture bleeding will help minimize complications related to the fracture.

The implants, in case of fracture requiring formal fracture surgery, do allow such surgery, without creating excessive difficulty for the treating surgeon.

The implants may be placed with minimally invasive technique, for example, using:
a) imaging modalities such as intra-operative X-ray, fluoroscopy or image intensification, CT, MRI, Ultrasound, PET scan;
b) intra-operative navigation systems which use calibration to known patient reference points to track movement of marked devices, such as, but not limited to, devices based on infra red light, (electro-) magnetic field, radiofrequency systems; and/or
c) appropriate instrumentation to allow reliable component placement with minimal morbidity from the procedure.

### Alternative Embodiment

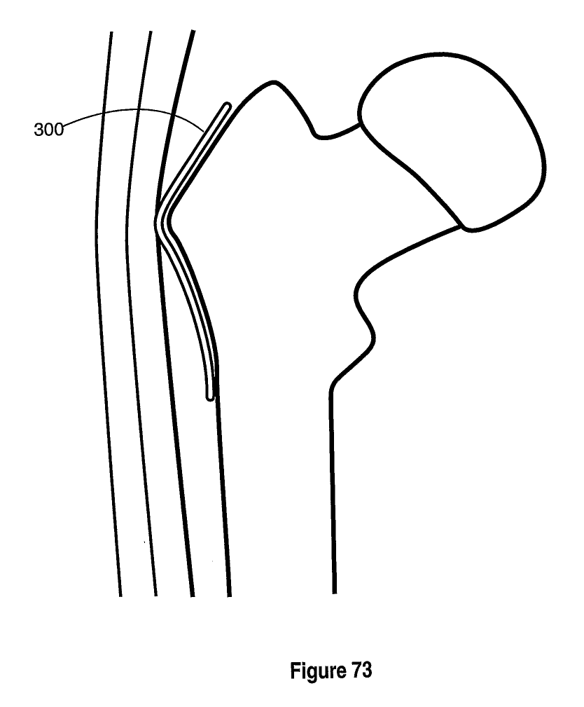

FIGS. 73 through 93 illustrate a second embodiment of an implant according to the invention.

As seen in FIG. 73 implant 300 may be placed directly adjacent to the bone, deep to the tendinous insertion of the abductor muscles and the origin of the vastus lateralis muscle.

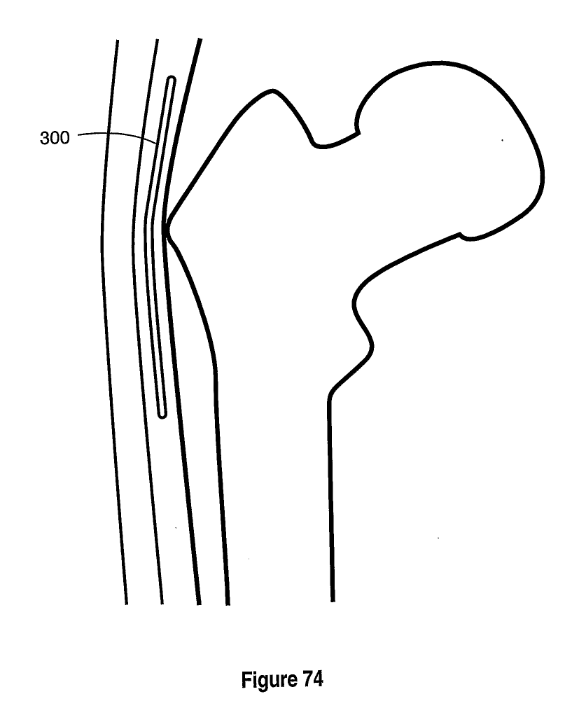

As seen in FIG. 74 implant 300 may be placed superficial to the tendinous insertion of the abductor muscles/origin of the vastus lateralis muscle and deep to the fascia lata.

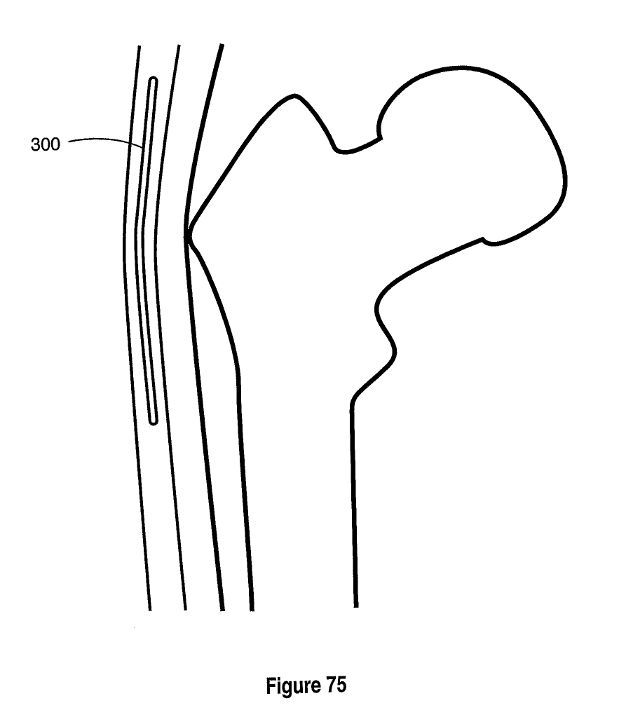

As seen in FIG. 75, implant 300 may be placed superficial to the fascia lata, deep into the subcutaneous tissues.

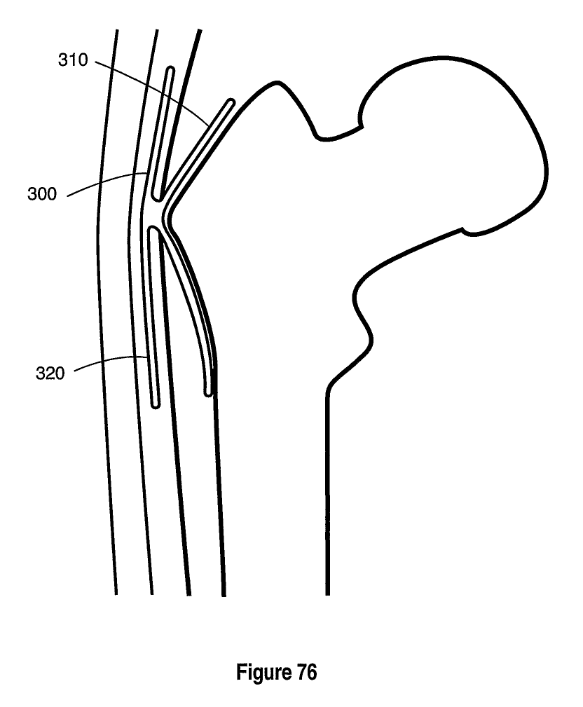

As seen in FIG. 76, implant 300 may include a first component 310 deep to the abductor tendon/origin of vastus lateralis and a second component 320 superficial to abductor tendon/origin of vastus lateralis and deep to fascia lata, the first and second components connected.

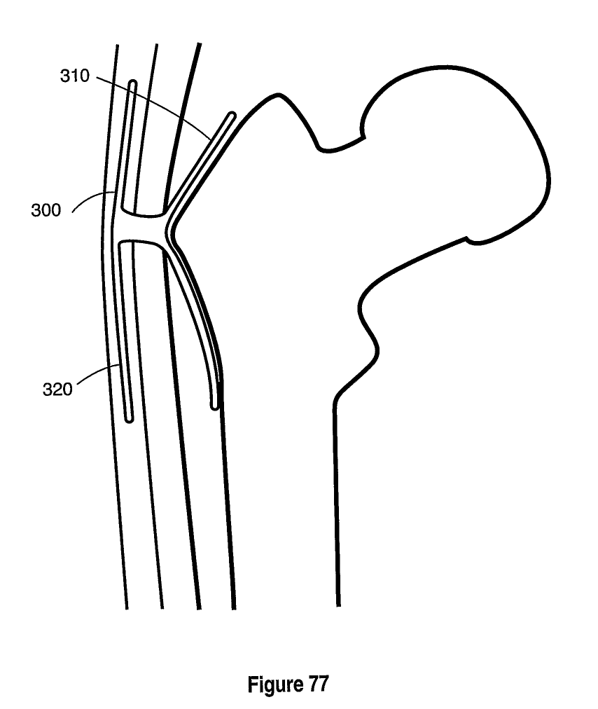

As seen in FIG. 77, implant 300 may include a first component 310 deep to abductor tendon/origin of vastus lateralis and a second component 320 superficial to the fascia lata, the first and second components connected.

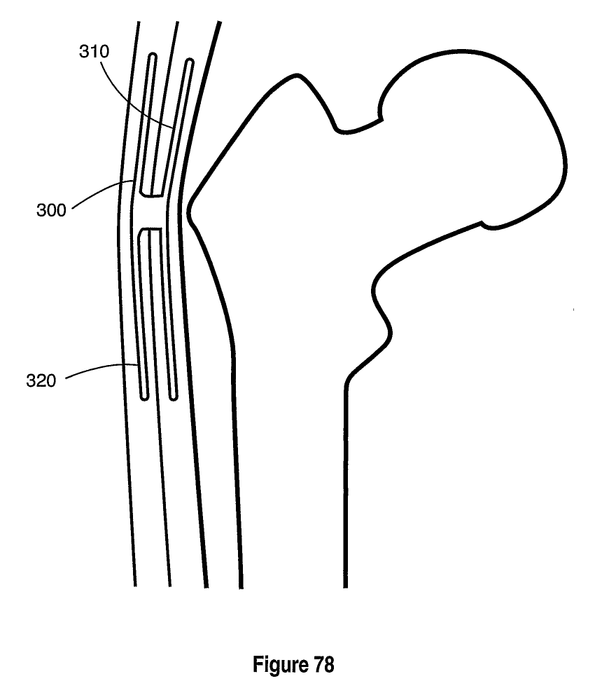

As seen in FIG. 78, implant 300 may include a first component 310 superficial to abductor tendon/origin of vastus lateralis and deep to the fascia lata, and a second component 320 superficial to the fascia lata, the first and second components connected.

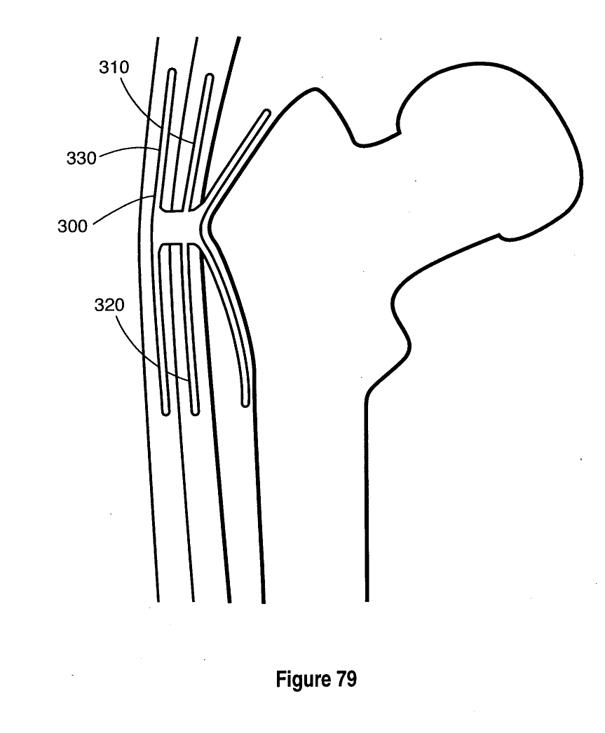

As seen in FIG. 79, the implant may include a first component 310 deep to the abductor tendon/origin of vastus lateralis, a second component 320 superficial to the abductor tendon/origin of vastus lateralis and deep to the fascia lata, and a third component 330 superficial to the fascia lata, the first, second and third components connected.

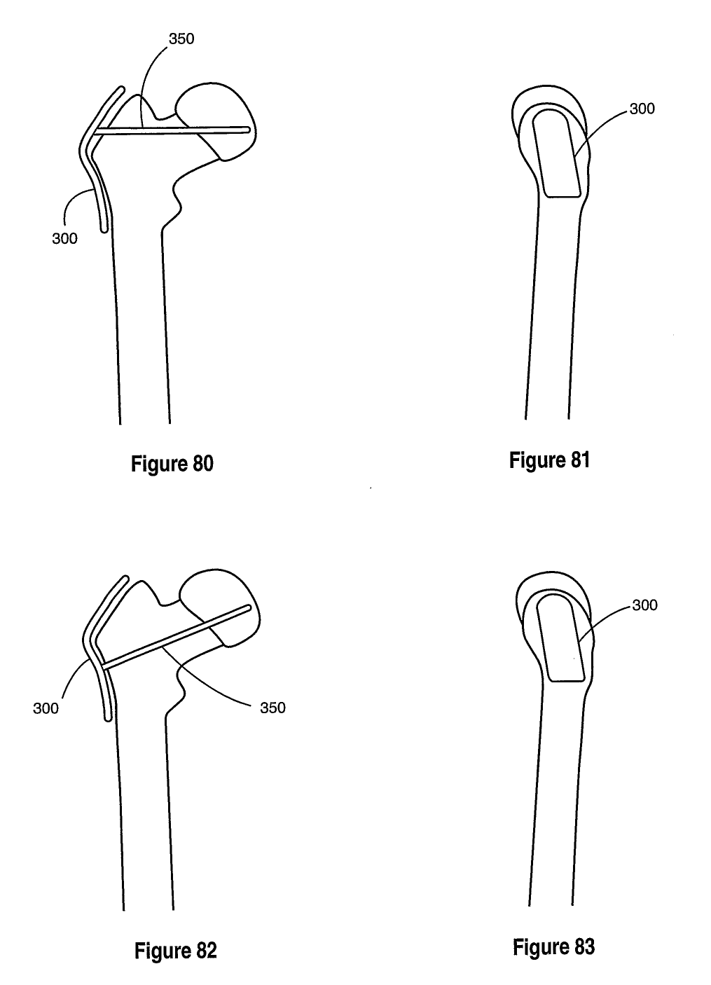

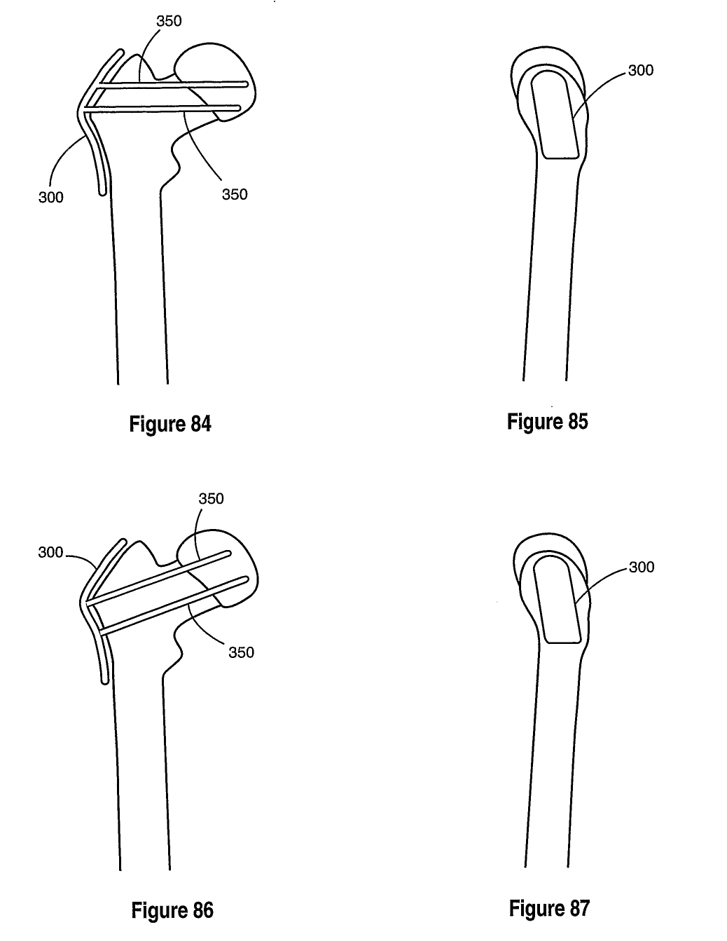

As seen in FIGS. 80 through 91, implant 300 may be connected to a one or more other implant placed into femoral neck 15. FIGS. 80 and 81 show implant 300 connected to a single second implant 350 implant placed into femoral neck 15, with the long axis of implant 350 perpendicular to the long axis of femur 1, in a plane coronal to the femoral neck 15, midway between the anterior and posterior border thereof. FIGS. 82 and 83 show a single second implant 350 placed into the femoral neck 15, with the long axis of the implant 350 parallel to the long axis of the femoral neck 15. FIGS. 84 and 85 show a plurality of additional implants 350 placed into femoral neck 15, with the long axis of implants 350 perpendicular to the long axis of femur 1. FIGS. 86 and 87 show a plurality of additional implants 350 placed into the femoral neck 15, with the long axis of implants 350 parallel to the long axis of femoral neck 15.

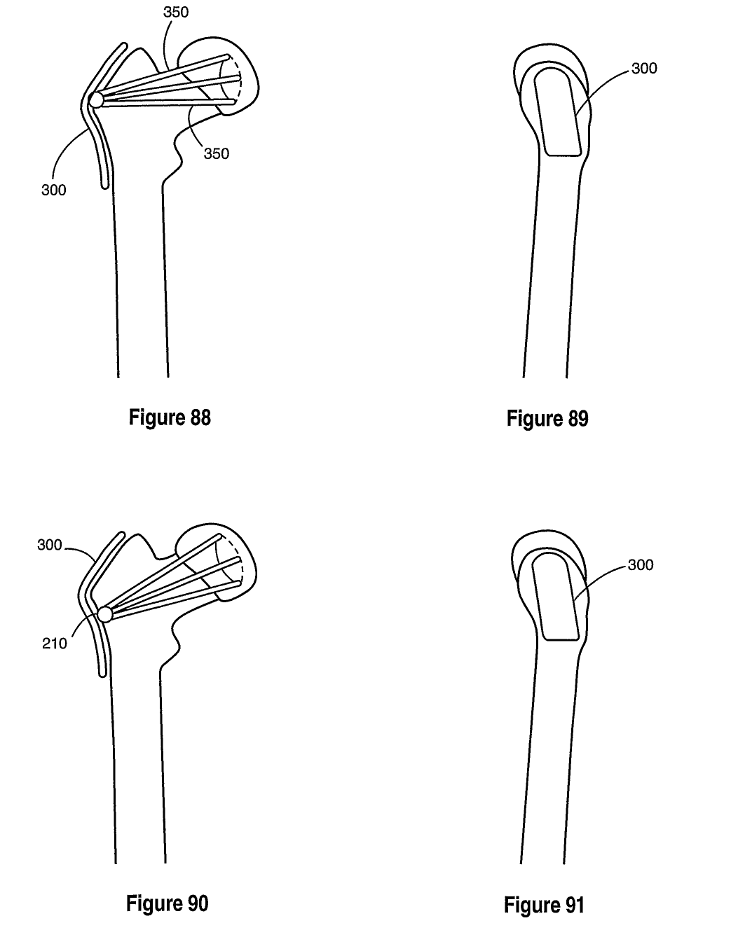

As seen in FIGS. 88 and 89 multiple additional implants 350 may be placed into femoral neck 15 through a single opening 210 with implants 350 forming a partial cone, and with the long axis of the cone approximately perpendicular to the long axis of femur 1, in a plane coronal to the femoral neck 15, midway between its anterior and posterior border. FIGS. 90 and 91 show a plurality of additional implants 350 placed into femoral neck 15 through a single opening 210, with the implants 350 forming a partial cone, and with the long axis of the cone parallel to the long axis of the femoral neck 15.

Implant 300 may extend distally and be intimately connected with the more distal femur, thus extending the area of protection against fracture to the subtrochanteric area and beyond.

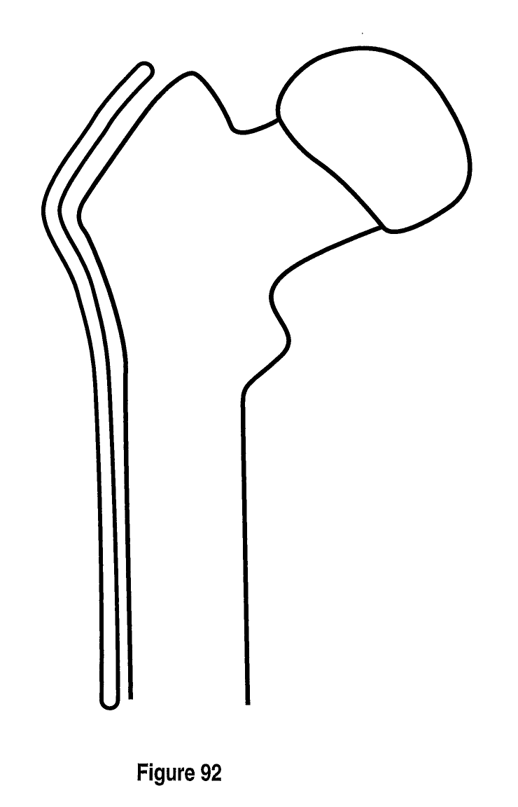

FIG. 92 is a view of implant 300 extending distally and intimately connected with the more distal femur;

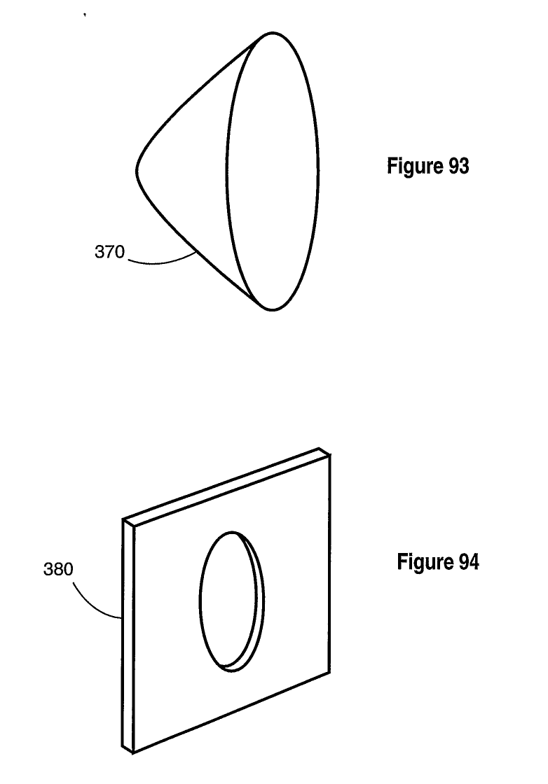

FIG. 93 shows a shallow cone implant 370. The concavity optimizes congruence with the protuberance of the greater trochanter and the adjacent bone.

FIG. 94 shows a raised rectangle implant 380, the corners of which may be rounded, with a medial concavity designed to optimize congruence with the protuberance of the greater trochanter and the adjacent bone.

Implant 300, according to the invention, may be placed as a single implant lateral to the area of the greater trochanter of the femur, sufficiently congruent with the lateral aspect of the femur to effectively increase the contact area of the proximal femur at the time of a fall. The increased contact area will lead to lower peak pressures and peak stresses, minimizing the risk of fracture of the proximal femur as the result of a fall. Implant 300 may extend beyond the borders of the bone, and the edges may be tapered. This differs from metallic implants for placement over the lateral aspect of the proximal femur in the area of the greater trochanter for fixation of greater trochanteric fracture or osteotomy, which aim to provide fixation, which is stable enough to allow bone healing.

Implant 300 may be placed directly adjacent to the bone, deep to the tendinous insertion of the abductor muscles and the origin of the vastus lateralis muscle. Congruency is dependent on the design of implant 300, compliance of implant 300, extent and compliance of the interface between implant 300 and the lateral aspect of the femur, such as material(s) used for fixation or fibrous tissue, formed secondarily as a response to the presence of implant 300.

Implant 300 may be placed deep to the fascia lata and superficial to the tendinous insertion of the abductor muscles and the origin of the vastus lateralis. Congruency is dependent on the design of implant 300, compliance of implant 300, extent and compliance of the interface between implant 300 and the lateral aspect of the femur, such as material(s) used for fixation, fibrous tissue formed secondarily as a response to the presence of implant 300, and the tendinous insertion of the abductor muscles/origin of the vastus lateralis.

Alternatively, implant 300 may be placed deep to the subcutaneous tissues and superficial to the fascia lata. Congruency is dependent on the design of the implant 300, compliance of the implant 300, extent and compliance of the interface between the implant 300 and the lateral aspect of the femur, such as material(s) used for fixation, fibrous tissue formed secondarily as a response to the presence of the implant 300, the tendinous insertion of the abductor muscles/origin of the vastus lateralis and the fascia lata.

Implant may also be placed at any two positions as described above, or in all three positions. Implant 300 may have interconnections between the components placed at different positions, or may have been placed as separate implant components.

Implant 300 may be connected to another implant or other implants placed in the femoral neck, providing mechanical reinforcement of the femoral neck, thus further decreasing the risk of fracture of the proximal femur at the time of a fall. The configuration of this combination of implants may resemble the configuration of rigid metallic constructs, which are commonly used in the treatment of fractures of the proximal femur, which aim to provide sufficiently rigid stability to allow bone healing.

Implant may extend distally and be intimately connected with the more distal femur, thus extending the area of protection against fracture to the subtrochanteric area and beyond. This plate further increases the contact area upon impact, as described above. In addition, this plate provides mechanical reinforcement, while remaining sufficiently compliant for it to act as a load sharing device, thereby allowing ongoing loading of the surrounding bone, sufficiently to minimize stress shielding of the bone. This increases the overall strength of the region of the femur covered by the implant 300. The configuration of this implant 300 may resemble the configuration of rigid metallic constructs, which are commonly used in the treatment of fractures of the proximal femur, which aim to provide sufficiently rigid stability to allow bone healing.

Implant 300 may have elastomeric qualities, leading to an, at least partially, elastic response to the impact from a fall, allowing significant initial absorption of energy, leading to a decrease in energy transfer to the femur, thus reducing the risk of fracture. If concurrent plastic deformation occurs, the implant 300 will become less able to absorb energy associated with repeated falls.

The implant(s) may have a solid core. Alternatively, the core of the implant may be hollow. The hollow core can be filled with non-biological substances, such as a watery liquid, such as physiological saline solution, or viscous or gelatinous substances, such as, but not limited to, silicone. Alternatively, the hollow implant can be filled with biological substances, such as fibrous tissue, adipose tissue, muscle, fascia, blood. Biological substances can be placed inside the hollow implant at or before the time of implantation, or can be allowed to fill the implant secondarily after implantation through conduits into the implant.

The implant may have the shape of a shallow cone (or part thereof), a medially concave, raised, (rounded) rectangle (or part thereof), a medially concave, raised, (rounded) polygon (or part thereof), or a relatively flat structure of which the shape of the medial side is determined at least in part by the anatomy of the lateral femur in the area of the greater trochanter and its associated soft tissues, with rounded anterior, posterior, proximal and distal borders, and the shape of its lateral side determined at least in part by the soft tissues lateral to the implant.

The implants may be made, at least partially, out of a non-porous metal alloy as commonly used in orthopaedic surgery, such as, but not limited to, cobalt chrome alloy, stainless steel, titanium alloy. Alternatively, the implants may be made, at least partially, Out of porous metals, such as, but not limited to, trabecular or porous tantalum, and trabecular or porous alloys, such as certain titanium-nickel formulations.

Also, the implants may be made, at least partially, out of synthetic materials such as, but not limited to, polyethylene of varying molecular weight and varying degree of cross-linking, polyurethane of varying composition, poly-aryl-ether-ketone and poly-ether-ether-ketone of varying composition, and/or polymethylmethylacrylate. These implants may be formed and provided with final shape, strength and stiffness prior to implantation, or they may form and achieve final shape, strength and stiffness after implantation. The latter is particularly applicable to selfcuring materials, either cold curing, such as, but not limited to, certain polyurethanes, or hot curing (exothermic reaction), such as polymethylmethacrylate. Certain thermoplastics may allow final shape adjustments to be made upon application of external heat to the implant, at the time of implantation.

The implants may furthermore be made, at least partially, out of a mineral substrate such as, but not limited to, coral, hydroxy-apatite, calcium phosphate formulations, human bone or derivatives, animal bone or derivatives, synthetic bone or derivatives, zirconia and alumina ceramics as commonly used in orthopaedic surgery, porous and/or polycrystalline silicon.

The implants, yet further, may be made at least partially, out of non-mineral biological material, such as human or animal cartilage, fibro-cartilage, muscle, fascia, adipose tissue, or derivatives. The human materials can be derived as autogenous or allogenous materials, directly harvested and subsequently processed, or as a result of bio-engineering processes and in-vitro or in-vivo tissue culture, including, but not limited to, the use of stem cells.

The implants may be made out of a composite of materials, such as, but not limited to, described above.

The implant may be in a secured in position, at least partially, through threading of at least part of the implant. Alternatively, the implant may be secured in position, at least partially, through the use of screws or pegs.

Other methods of securing the implant, include, at least partially, through a relatively thin layer of appropriately formulated polymethylmethacrylate (commonly known as ‘bone cement’), not unlike the fixation of cemented joint replacement components. Also the implant may be secured in position, at least partially, by bony or soft tissue ingrowth and/or ongrowth, as may be induced or conducted by the surgery itself, the compliant, non-rigid nature of the implant and/or its fixation method, or by the characteristics of the surface of the implant, such as, but not limited to, the presence of pores, beads, crevices, coating such as plasma spray, hydroxy apatite or calcium phosphate formulations, the presence of biologically active agents, such as bone or soft tissue metabolism modulating agents, vectors or inductors for gene therapy, enzymes or catalysts. Temporary fixation may be provided initially through design features such as, but not limited to, the use of sutures, absorbable or non-absorbable, and/or screws/pegs/hooks/barbs/suture anchors, absorbable or non-absorbable.

The implant may furthermore be secured in position, at least partially, by implant geometry that resists motion into the direction opposite to insertion, such as, but not limited to, the following: the presence of flexible barbs; or implant geometry with the diameter of part of the implant greater than the residual diameter of an opening created in the femoral cortex/fascia lata/abductor insertion for introduction of the implant, facilitated by introduction of the implant in liquid or viscous form, such as applicable to certain self-curing synthetic materials as described above, use of sufficient force in the direction of the long axis of the implant fixation feature to induce an elastic response in the implant, leading to a transient decrease in its diameter, sufficient to allow implant introduction, followed by expansion to, or close to, the initial pre-insertion diameter.

The implants, in case of fracture requiring formal fracture surgery, do allow such surgery without excessive difficulty for the treating surgeon.

Although the particular preferred embodiments of the invention have been disclosed in detail for illustrative purposes, it will be recognized that variations or modifications of the disclosed apparatus lie within the scope of the present invention.

1. An implant system for a femur, the femur having a femoral neck, a femoral head and a lateral femoral cortex, comprising, an implant placeable inside said femoral neck through an opening in said lateral femoral cortex, said implant extending from said opening to a position within said femoral head.

2. The implant system of claim 1 further comprising a second implant starting from said opening to a second position within said femoral head.

3. The implant system of claim 2 further comprising a third implant, said third implant extending from said opening to a third position in said femoral head, said first, second and third implants forming a partial cone.

4. The implant system of claim 1 further comprising a second implant, said second implant extending from a second opening in said lateral femoral cortex to a second position within said femoral head.

5. The implant system of claim 1 wherein said implants are curved.

6. The implant system of claim 1, said femur having a greater trochanter, wherein said implant system further comprises a plate in the area of said greater trochanter.

7. An implant system for a femur, said femur having a lateral aspect, said implant approximately congruent to the lateral aspect of said femur.

8. The implant system of claim 7 wherein said implant is placed adjacent to said femur, deep to the tendinous insertion of abductor muscles.

9. The implant system of claim 7 wherein said implant is placed deep to the subcutaneous tissues and superficial to the fascia lata.

10. The implant system of claim 7 wherein said implant is placed deep to the fascia lata and superficial to the tendinous insertion of abductor muscles.

11. The implant system of claim 7, wherein said implant comprises a first component connected to a second component, said first component positioned in a plane approximately parallel to said second component.

12. The implant system of claim 11 wherein said implant has elastomeric qualities.

13. The implant system of claim 2 wherein said implants are curved.

14. The implant system of claim 2, said femur having a greater trochanter, wherein said implant system further comprises a plate in the area of said greater trochanter.

15. The implant system of claim 3 wherein said implants are curved.

16. The implant system of claim 3, said femur having a greater trochanter, wherein said implant system further comprises a plate in the area of said greater trochanter.

17. The implant system of claim 4 wherein said implants are curved.

18. The implant system of claim 4, said femur having a greater trochanter, wherein said implant system further comprises a plate in the area of said greater trochanter.
